(19) United States
(12) Patent Application Publication
(10) Pub. No.: US 2025/0205258 A1
(43) Pub. Date: Jun. 26, 2025

(54) DERIVATIVES OF BUFALIN AND THEIR PREPARATION METHODS, AS WELL AS COMPOSITIONS, PREPARATIONS, AND APPLICATIONS

(71) Applicant: SHANGHAI MEWSGEN BIOTECHNOLOGY CO., LTD, Shanghai (CN)

(72) Inventors: Juan WANG, Shanghai (CN); Yulan MIAO, Shanghai (CN)

(21) Appl. No.: 18/979,485

(22) Filed: Dec. 12, 2024

Related U.S. Application Data
(63) Continuation of application No. PCT/CN2023/139821, filed on Dec. 19, 2023.

(30) Foreign Application Priority Data
Dec. 22, 2022 (CN) 202211654524.3
Jul. 18, 2023 (CN) 202310879936.5

(51) Int. Cl. A61K 31/675; A61P 35/00; A61P 35/04; C07J 43/00

(52) U.S. Cl. CPC A61K 31/675; A61P 35/00; A61P 35/04; C07J 43/003

(57) ABSTRACT

The present invention discloses a novel series of bufalin derivatives and their pharmaceutically acceptable salts, obtained by introducing different phosphate ester groups into the structure of existing bufalin. These derivatives not only exhibit excellent antitumor activity and lower hERG potassium channel inhibitory activity, but also significantly reduce toxic and side effects. They can be used for the preparation of various antitumor drugs, therapeutic agents for cardiovascular and cerebrovascular diseases, as well as therapeutic agents for neurological diseases. The bufalin derivatives provided by this invention come in a variety of structural forms and their pharmaceutically acceptable salts, meeting the needs for preparing multiple drug compositions and pharmaceutical formulations. They have broad application prospects. Furthermore, their preparation method is simple and convenient, with low-cost and easily obtainable raw materials, diverse options, and ease of industrial production. Therefore, they have great value for promotion and application.

### CROSS-REFERENCE TO RELATED APPLICATIONS

This application is a continuation of International Patent Application No. PCT/CN2023/139821, filed on Dec. 19, 2023, which claims the benefit of priority from Chinese Patent Application No. 202211654524.3, filed on Dec. 22, 2022; and Chinese Patent Application No. 202310879936.5, filed on Jul. 18, 2023. The content of the aforementioned applications, including any intervening amendments thereto, is incorporated herein by reference.

### FIELD OF THE INVENTION

The present invention relates to derivatives of bufalin, particularly to derivatives of bufalin and their preparation methods, as well as compositions, preparations, and applications thereof, belonging to the field of pharmaceutical technology.

### BACKGROUND OF THE INVENTION

Bufalin, derived from the traditional Chinese medicine Chan Su, is a bufadienolide compound possessing various biological activities such as analgesia, cardiotonic effects, and antitumor properties. Its chemical structure is

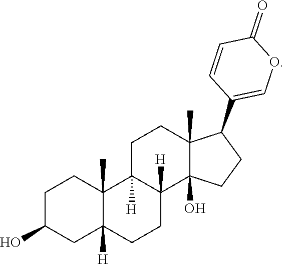

Research has found that bufalin can significantly inhibit the expression of liver cancer stem cell markers CD133, CD44, and ESA, demonstrating its effect on inhibiting liver cancer stem cells (Chinese Journal of Information on Traditional Chinese Medicine, 2021, 12: 41-44). Additionally, bufalin inhibits the proliferation of melanoma A375 cells in a concentration- and time-dependent manner, significantly increasing the activity of Caspase-9 and Caspase-3 proteins within A375 cells, enhancing the apoptosis of A375 cells, and arresting the cell cycle at the S phase (Journal of Youjiang Medical University for Nationalities, 2021, 6: 719-724). Studies have also shown that bufalin can inhibit the proliferation of ovarian cancer cells SK—OV-3 in a concentration- and time-dependent manner through the EGFR/AKT/ERK signaling pathway (Dou L, et al., Chin Med J (Engl) (2021) 135(4):456-461). By inhibiting the STAT3 signaling pathway, bufalin can also suppress tumor microenvironment-mediated angiogenesis (Kai F, et al., J Transl Med (2021) 19(1):383). Research has further discovered that bufalin induces the death of neuroblastoma cells U87 through both apoptosis and necrosis (Hai Rui L H, et al., Onco Targets Ther (2020) 13:4767-4778). Despite bufalin's excellent antitumor activity, its relatively high toxicity and narrow therapeutic index necessitate structural modification to enhance the compound's activity and reduce toxicity, potentially leading to the discovery of new antitumor drugs. Moreover, bufalin exhibits significant cardiotoxicity, which limits its development as a pharmaceutical agent (Min L, et al., Chin J Nat Med (2020) 18(7):550-560). Therefore, synthesizing bufalin derivatives and studying their structure-activity relationships to discover bufalin derivatives with high efficacy and low toxicity is of great significance. Currently, several patent documents have disclosed technical solutions based on the structural modification of bufalin and its applications. For example: WO201185641A1 discloses a class of bufalin derivatives and their use in treating cancer. CN102532235 discloses a class of bufadienolide derivatives and their preparation methods, as well as the use of compositions containing these derivatives. Furthermore, patent documents such as CN102656179, CN103980337, CN110483608, and CN103980338 each disclose technical solutions for a class of bufalin derivatives, their pharmaceutical compositions, and uses. Their main contribution is the esterification of the 3-hydroxy group of bufalin to obtain a series of derivatives. Obviously, current research is still not sufficiently in-depth and requires continued related research and development to better contribute to safeguarding people's physical and mental health.

### SUMMARY OF THE INVENTION

Based on the aforementioned objectives, the present invention firstly provides a novel bufalin derivative and its preparation method. Furthermore, based on this, the invention also provides pharmaceutical compositions, formulations, and applications of the said bufalin derivative. To achieve the aforementioned objectives, the present invention firstly provides a bufalin derivative:

The bufalin derivative is a compound with a structure represented by Formula I, Formula II, or Formula III, as well as pharmaceutically acceptable salts:

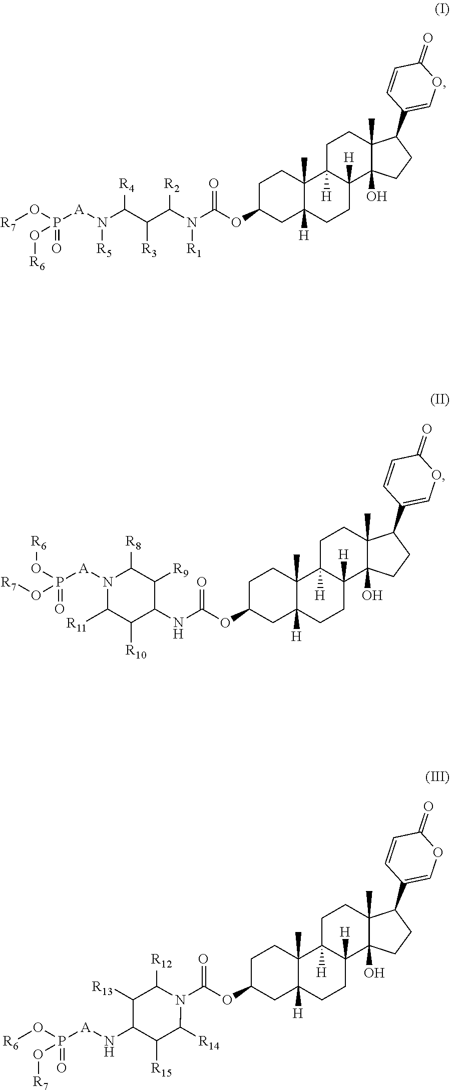

Wherein: R1 is selected from any one of hydrogen, deuterium, and substituted or unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl;
R2 and R3 are each independently selected from any one of hydrogen, deuterium, hydroxyl, halogen, and substituted or unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl;
   R4 and R1 together form a substituted or unsubstituted nitrogen-containing heterocyclic ring;
   R5 is selected from any one of hydrogen, deuterium, and substituted or unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl, or R5 and R1 together form a substituted or unsubstituted nitrogen-containing heterocyclic ring;
   R6 and R7 are each independently selected from any one of hydrogen, and substituted or unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl.
R8, R9, R10, and R11 are each independently selected from any one of hydrogen, deuterium, hydroxyl, halogen, and substituted or unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl;
   R12, R13, R14, and R15 are each independently selected from any one of hydrogen, deuterium, hydroxyl, halogen, and substituted or unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl.
A is a linear or branched alkane containing C1 to C6.
   Preferably, in the compounds and their pharmaceutically acceptable salts having the structure of Formula II or Formula III:
   R6 and R7 are each independently selected from 3 to 8-membered cycloalkyl, C6 to C12 aryl, and any one of C1 to C₈ alkyl, 3 to 8-membered cycloalkyl C1 to C8 alkyl, 3 to 8-membered heterocyclyl C1 to C8 alkyl, 3 to 8-membered alkyl containing an ether bond, or thioether bond, each optionally substituted with one or more T groups;
R8, R9, R10, and R11 are each independently selected from 3 to 8-membered cycloalkyl, 3 to 8-membered alkyl containing a thioether bond, and any one of C1 to C8 alkyl, 3 to 8-membered cycloalkyl C1 to C8 alkyl, 3 to 8-membered heterocyclyl C1 to C₈ alkyl, 3 to 8-membered alkyl containing an ether bond, or thioether bond, each optionally substituted with one or more T groups;
   R12, R13, R14, and R15 are each independently selected from 3 to 8-membered cycloalkyl, 3 to 8-membered alkyl containing a thioether bond, and any one of C1 to C8 alkyl, 3 to 8-membered cycloalkyl C1 to C8 alkyl, 3 to 8-membered heterocyclyl C1 to C8 alkyl, 3 to 8-membered alkyl containing an ether bond, or thioether bond, each optionally substituted with one or more T groups;
   The T group is selected from any one of the groups F, Cl, Br, I, OH, OCH₃, OCH₂CH₃, SCH₃, SCH₂CH₃, NHBoc, NHC(═O)CH₃, NHC(═O)CH₂CH₃, C(═O)NH₂, C(═O)OC(CH₃)₃, C(═O)OCH₂CH₃, CH₂F, CHF₂, CF₃.

Further preferably, in the compounds and their pharmaceutically acceptable salts having the structure of Formula II or Formula III:

R6 and R7 are each independently selected from any one of 3 to 8-membered cycloalkyl and C6 to C12 aryl, each optionally substituted with one or more T groups;
R8, R9, R10, and R11 are each independently selected from 3 to 8-membered alkyl containing an ether bond, each optionally substituted with one or more T groups; R12, R13, R14, and R15 are each independently selected from 3 to 8-membered alkyl, each optionally substituted with one or more T groups;
The T group is selected from any one of the groups F, Cl, Br, I, OH, OCH₃, OCH₂CH₃, SCH₃, SCH₂CH₃, NHBoc, NHC(═O)CH₃, NHC(═O)CH₂CH₃, C(═O)NH₂, C(═O)OC(CH₃)₃, C(═O)OCH₂CH₃, CH₂F, CHF₂, CF₃.

Even more preferably the bufalin derivatives are:

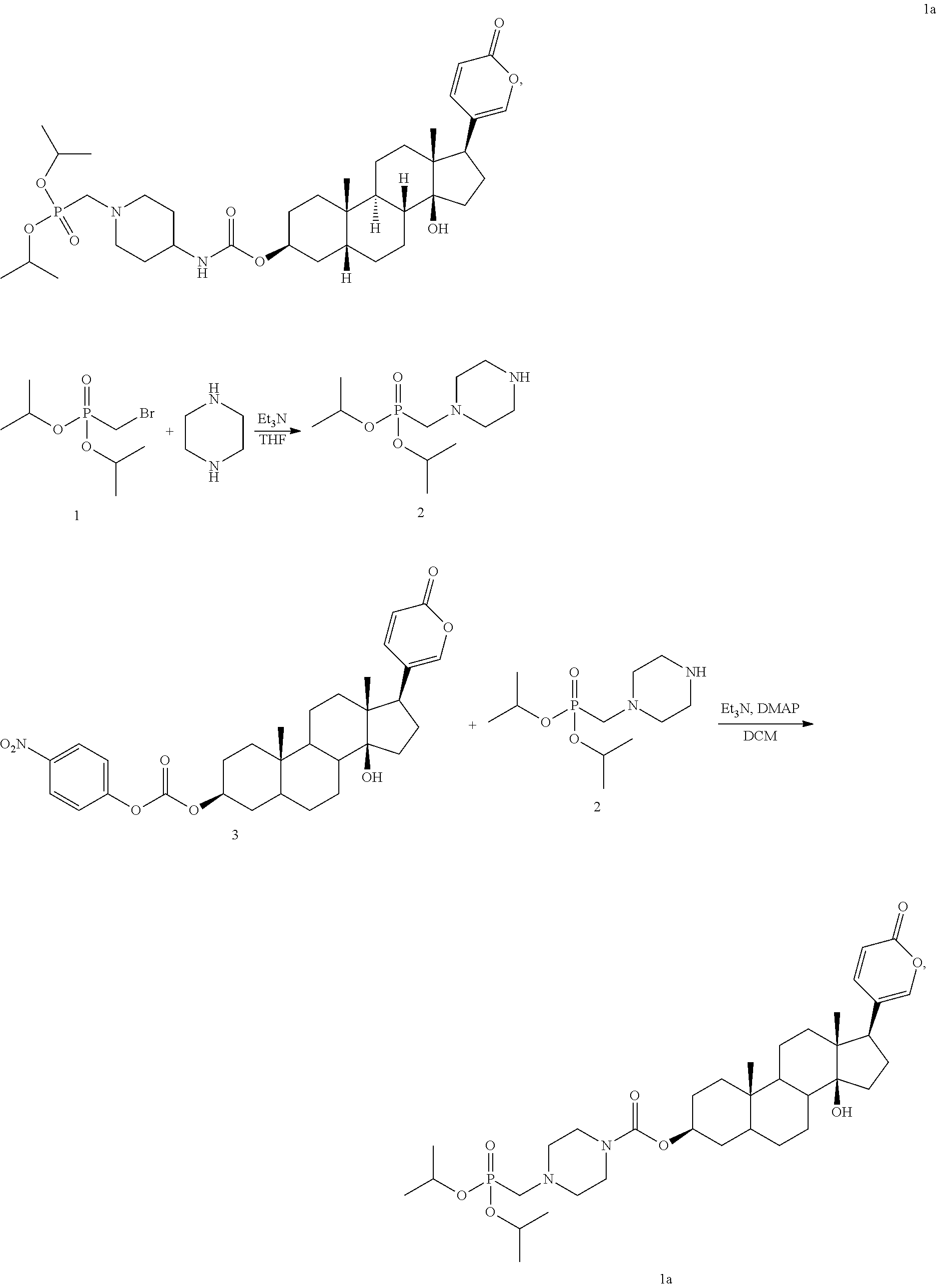

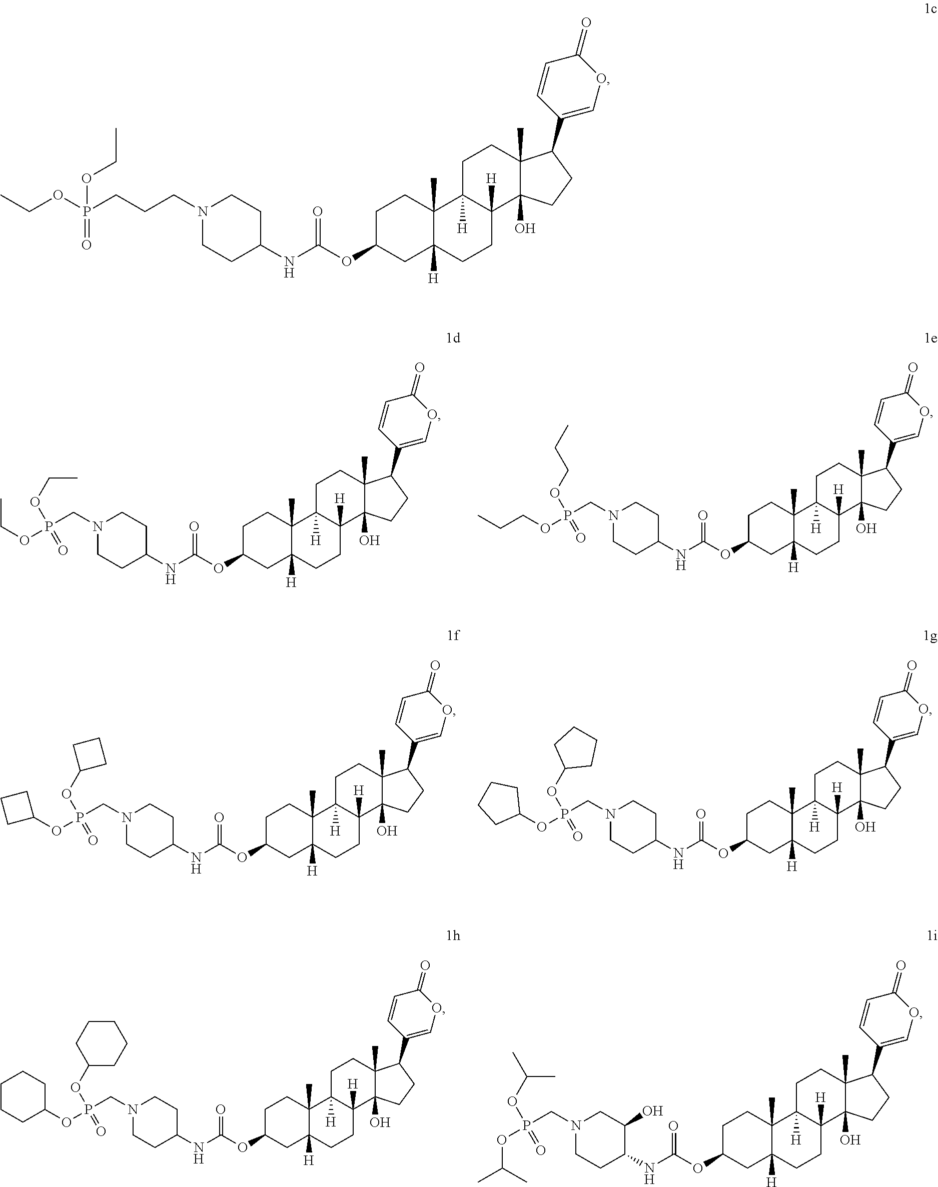

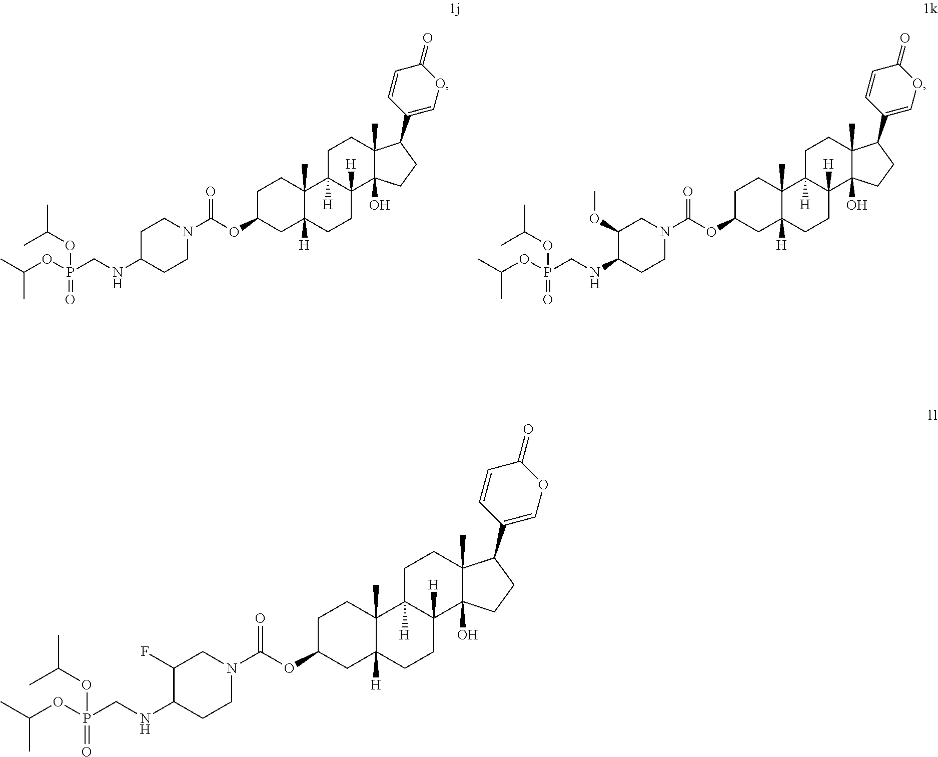

Compounds having the structures shown in Formulas 1a to 11 and their pharmaceutically acceptable salts.

Furthermore, the pharmaceutically acceptable salts of the compounds are formed by reacting the compounds with a pharmaceutically acceptable inorganic acid or organic acid;

Wherein: the inorganic acid is any one of hydrochloric acid, hydrobromic acid, phosphoric acid, nitric acid, or sulfuric acid;

The organic acid is any one of formic acid, acetic acid, propionic acid, succinic acid, 1,5-naphthalenedisulfonic acid, asiatic acid, glycyrrhizic acid, glycyrrhetic acid, oleanolic acid, crataegic acid, ursolic acid, corosolic acid, betulinic acid, boswellic acid, oxalic acid, tartaric acid, lactic acid, salicylic acid, benzoic acid, valeric acid, diethylacetic acid, malonic acid, succinic acid, fumaric acid, pimelic acid, adipic acid, maleic acid, malic acid, aminosulfonic acid, phenylpropionic acid, gluconic acid, ascorbic acid, nicotinic acid, isonicotinic acid, methanesulfonic acid, ethanesulfonic acid, p-toluenesulfonic acid, citric acid, or an amino acid.

Secondly, the present invention also provides a method for preparing the bufalin derivatives having the structure of Formula I mentioned above. The preparation method includes step e, or steps a to b and e, or steps c to d and e in the following synthesis route:

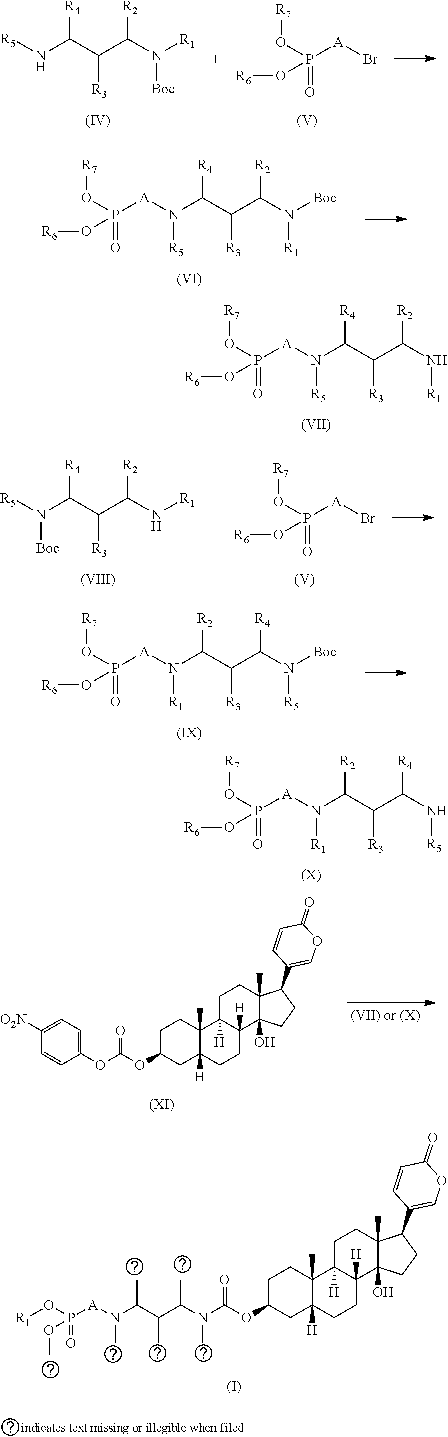

Wherein: in step a, compound IV reacts with compound V in an organic solvent containing a first alkaline catalyst to produce compound VI, and the molar ratio of compound IV to compound V to the first alkaline catalyst is 1: (1-3): (1-3);
In step b, compound VI is catalyzed by an acidic catalyst in an organic solvent to produce compound VII;
In step c, compound VIII reacts with compound V in an organic solvent containing a first alkaline catalyst to produce compound IX, and the molar ratio of compound VIII to compound V to the first alkaline catalyst is 1: (1-3): (1-3);
In step d, compound IX is catalyzed by an acidic catalyst in an organic solvent to produce compound X;
In step e, compound XI reacts with compound VII or compound XI reacts with compound X respectively in an organic solvent containing a second alkaline catalyst to produce compound I, and the molar ratio of compound XI to compound VII to the second alkaline catalyst or the molar ratio of compound XI to compound X to the second alkaline catalyst is 1: (1-5): (1-5) respectively.

Further details: The first alkaline catalyst is an inorganic alkaline compound, the second alkaline catalyst is a nitrogen-containing compound, and the acidic catalyst is an inorganic acid or an organic acid.

Optionally: The inorganic alkaline compound is any one or a mixture of more than one of sodium carbonate, potassium carbonate, or cesium carbonate;
The nitrogen-containing compound is any one or a mixture of more than one of 4-dimethylaminopyridine, triethylamine, or pyridine;
The acidic catalyst is any one of hydrochloric acid, sulfuric acid, trifluoroacetic acid, acetic acid, or formic acid;
The organic solvent is any one or a mixture of more than one of dichloromethane, dichloroethane, ethyl acetate, acetonitrile, or tetrahydrofuran.

Furthermore, the present invention also provides a pharmaceutical composition of the aforementioned bufalin derivatives:

The pharmaceutical composition contains a therapeutically effective amount of the aforementioned bufalin derivatives or a pharmaceutically acceptable salt of the bufalin derivatives, and further contains one or more of pharmaceutically acceptable carriers, excipients, and adjuvants.
Furthermore, the present invention also provides a pharmaceutical formulation of the aforementioned bufalin derivatives:
The pharmaceutical formulation is at least one of an injection, a powdered injection, an emulsion for injection, a tablet, a pill, a capsule, a paste, a cream, a patch, a liniment, a powder, a spray, an implant, a drop, a suppository, an ointment, or a nano-formulation prepared using the aforementioned pharmaceutical composition, wherein:
The injection is at least one of a small volume injection, a medium volume injection, or a large volume injection, and the nano-formulation is a liposome.

Furthermore, the present invention also provides an application of the aforementioned bufalin derivatives:

The application is the use of a therapeutically effective amount of the aforementioned bufalin derivatives or a pharmaceutically acceptable salt of the bufalin derivatives for preparing at least one of an anti-tumor drug, a drug for treating cardiovascular and cerebrovascular diseases, or a drug for treating neurological diseases, wherein:
The anti-tumor drug is used for treating at least one malignant tumor growing in the esophagus, stomach, intestine, rectum, mouth, pharynx, larynx, lungs, colon, breast, uterus, endometrium, ovaries, prostate, testes, bladder, kidneys, liver, pancreas, bones, connective tissues, skin, eyes, brain, and central nervous system of humans or animals, as well as at least one of thyroid cancer, leukemia, Hodgkin's disease, lymphoma, and myeloma; or
The application is the use of a therapeutically effective amount of the aforementioned bufalin derivatives or a pharmaceutically acceptable salt of the bufalin derivatives for preparing a drug that inhibits tumor metastasis.

The term “alkyl” used in the present invention refers to: a saturated, straight-chain or branched, monovalent hydrocarbon radical having from one to twelve carbon atoms, wherein the hydrogen atoms in the hydrocarbon radical can optionally be independently substituted by one or more substituents. Examples include, but are not limited to: methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, cyclopropyl, cyclobutyl, cyclopentyl, cyclohexyl, cycloheptyl, and cyclooctyl.

The term “alkenyl” used in the present invention refers to: a straight-chain or branched monovalent hydrocarbon radical having from two to twelve carbon atoms with at least one unsaturated position, i.e., a carbon-carbon sp² double bond, wherein the hydrogen atoms in the alkenyl group can optionally be independently substituted by one or more substituents, and also includes those having “cis” and “trans” orientations or “E” and “Z” configurations. Examples include, but are not limited to:

vinyl, allyl, 1-cyclopent-1-enyl, 1-cyclopent-2-enyl, 1-cyclopent-3-enyl, 5-hexenyl, 1-cyclohex-2-enyl, 1-cyclohex-2-enyl (note: this is repeated and should likely be 1-cyclohex-3-enyl or another isomer to avoid repetition), and 1-cyclohex-3-enyl.

The term “alkynyl” used in the present invention refers to: a straight-chain or branched monovalent hydrocarbon radical having from two to twelve carbon atoms with at least one unsaturated position, i.e., a carbon-carbon sp³ triple bond, wherein the hydrogen atoms in the alkynyl group can optionally be independently substituted by one or more substituents. Examples include, but are not limited to: ethynyl and propynyl.

The term “cycloalkyl” used in the present invention refers to: a monovalent non-aromatic saturated or partially saturated cyclic hydrocarbon radical having from three to ten carbon atoms. Examples include, but are not limited to: cyclopropyl, cyclobutyl, cyclopentyl, 1-cyclopent-1-enyl, 1-cyclopent-2-enyl, 1-cyclopent-3-enyl (note: these are alkenyl groups and should be excluded if only cycloalkyl is intended), cyclohexyl, 1-cyclohex-1-enyl, 1-cyclohex-2-enyl, 1-cyclohex-3-enyl, cyclohexadienyl, cycloheptyl, cyclooctyl, cyclodecyl, cycloundecyl, cyclododecyl; and also includes bicyclic, tricyclic, and more cyclic cycloalkyl structures, wherein the polycyclic structures optionally include saturated or partially unsaturated cycloalkyl or heterocyclic radicals or aryl or heteroaryl rings fused to the saturated or partially unsaturated cycloalkyl, wherein bicyclic carbon rings with 7 to 12 atoms can be arranged as bicyclo[4,5], [5,5], [5,6], or [6,6] systems, or as bridged systems including bicyclo[2.2.1]heptane, bicyclo[2.2.2]octane, and bicyclo[3.2.2]nonane. (Note: I have excluded the alkenyl groups from the cycloalkyl examples to maintain consistency with the term's definition.)

The term “heteroalkyl” used in the present invention refers to: a saturated straight-chain or branched monovalent hydrocarbon radical having from one to twelve carbon atoms, wherein at least one carbon atom in the hydrocarbon radical is replaced by a heteroatom selected from nitrogen, oxygen, or sulfur. The nitrogen, oxygen, or sulfur atom replacing at least one carbon atom in the hydrocarbon radical can appear in the middle or at the end of the radical, either as a carbon-based group or a heteroatom-based group. Furthermore, the hydrogen atoms on the hydrocarbon radical of the heteroalkyl can optionally be independently substituted by one or more substituents.

Additionally, the term “heteroalkyl” also encompasses alkoxy and heteroalkoxy groups.

The term “heteroalkenyl” used in the present invention refers to: a straight-chain or branched monovalent hydrocarbon radical having from two to twelve carbon atoms and containing at least one double bond. Examples include, but are not limited to, vinyl and allyl, wherein at least one carbon atom in the hydrocarbon radical is replaced by a heteroatom selected from nitrogen, oxygen, or sulfur. The nitrogen, oxygen, or sulfur atom replacing at least one carbon atom in the hydrocarbon radical can appear in the middle or at the end of the radical, either as a carbon-based group or a heteroatom-based group.

Furthermore, the hydrogen atoms on the hydrocarbon radical of the heteroalkenyl can optionally be independently substituted by one or more substituents, including substituents with “cis” and “trans” orientations or “E” and “Z” configurations.

The term “heteroalkynyl” used in the present invention refers to: a straight-chain or branched monovalent hydrocarbon radical having from two to twelve carbon atoms and containing at least one triple bond. Examples include, but are not limited to, ethynyl and propynyl, wherein at least one carbon atom in the hydrocarbon radical is replaced by a heteroatom selected from nitrogen, oxygen, or sulfur. The nitrogen, oxygen, or sulfur atom replacing at least one carbon atom in the hydrocarbon radical can appear in the middle or at the end of the radical, either as a carbon-based group or a heteroatom-based group. Additionally, the hydrogen atoms on the hydrocarbon radical of the heteroalkynyl can optionally be independently substituted by one or more substituents.

The terms “heterocyclyl” and “heterocyclic” used in the present invention have the same meaning and can be used interchangeably. They are defined as: a saturated or partially unsaturated carbocyclic group having from 3 to 8 ring atoms, wherein at least one ring atom is independently selected as a heteroatom from nitrogen, oxygen, or sulfur, and the remaining ring atoms are carbon atoms. Furthermore, one or more of these ring atoms can optionally be independently substituted by one or more substituent groups, which can be either carbon-based groups or heteroatom-based groups.

Furthermore, the term “heterocyclyl” also encompasses heterocyclylalkoxy groups and heterocyclyl rings fused with saturated, partially unsaturated, or fully unsaturated (i.e., aromatic) carbocyclic or heterocyclic rings. Examples of heterocyclyl rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidinyl, morpholinyl, 4-thiomorpholinyl, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, dihydroindolyl, 2H-pyranyl, 4H-pyranyl, dioxanyl, 1,3-dioxolanyl, pyrazolinyl, dithianyl, dihydropyranyl, dihydrothienyl, dihydrofuranyl, pyrazolidinyl, imidazolinyl, imidazolidinyl, 3-azabicyclo[3.1.0]hexane, 3-azabicyclo[4.1.0]heptane, azabicyclo[2.2.2]hexane, 3H-indolyl, quinazolinyl, and N-pyridinylurea. Additionally, spirocyclic moieties are also included within the scope of the term “heterocyclyl”.

Furthermore, the term “heterocyclyl” used in the present invention can be either C-linked or N-linked, as long as it is technically feasible. For example, a group derived from pyrrole can be N-linked pyrrol-1-yl or C-linked pyrrol-3-yl. Additionally, a group derived from imidazole can be N-linked imidazol-1-yl or C-linked imidazol-3-yl. Examples of heterocyclyl groups where two ring carbon atoms are substituted by oxo (C═O) moieties include dihydroisoindol-1,3-dionyl and 1,1-dioxothiomorpholinyl. These heterocyclyl groups can be unsubstituted or substituted at one or more substitutable positions with various technically feasible groups, as specified.

The term “aryl” used in the present invention refers to an optionally substituted monocyclic or polycyclic group or ring system containing at least one aromatic hydrocarbon ring, including but not limited to phenyl, naphthyl, fluorenyl, azulenyl, anthryl, phenanthryl, pyrenyl, biphenyl, and terphenyl.

The term “heteroaryl” used in the present invention refers to an optionally substituted monocyclic or polycyclic group or ring system containing at least one aromatic ring with one or more heteroatoms independently selected from nitrogen, oxygen, and sulfur. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridinyl, pyrimidinyl, pyrrolyl, thiadiazolyl, thiazolyl, thiophenyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothiophenyl, benzotriazolyl, benzoxazolyl, furano[2,3-b]pyridinyl, imidazo[1,2-a]pyridinyl, imidazo[2,1-b]thiazolyl, indolinyl, indolyl, indazolyl, isobenzofuranyl, isobenzothiophenyl, isoindolyl, isoquinolinyl, isothiazolyl, naphthyridinyl, oxazolo[4,5-b]pyridinyl, phthalazinyl, pteridinyl, purinyl, pyrido[2,3-b]pyridinyl, pyrrolo[2,3-b]pyridinyl, quinolinyl, quinoxalinyl, quinazolinyl, thiadiazolo[4,5-d]pyrimidinyl, and thiopheno[2,3-b]pyridinyl. Examples of tricyclic heteroaryl groups include, but are not limited to, acridinyl, benzoindolyl, carbazolyl, dibenzofuranyl, perimidinyl, phenanthrolinyl, phenanthridinyl, phenarsazinyl, phenazinyl, phenothiazinyl, phenoxazinyl, and xanthenyl.

The term “arylalkyl” used in the present invention refers to an alkyl group substituted by one or more aryl moieties as defined above, wherein the alkyl group is defined as per the above definition of alkyl. Examples of arylalkyl groups include aryl-C1-3-alkyl, such as but not limited to benzyl and phenylethyl.

The term “heteroarylalkyl” used in the present invention refers to an alkyl group substituted by a heteroaryl moiety as defined above, wherein the alkyl group is defined as per the above definition of alkyl. Examples of heteroarylalkyl groups include five- or six-membered heteroaryl-C1-3-alkyl, such as but not limited to oxazolylmethyl and pyridylethyl.

The term “heterocyclylalkyl” used in the present invention refers to an alkyl group substituted by a heterocyclyl moiety as defined above, wherein the alkyl group is defined as per the above definition of alkyl. Examples of heterocyclylalkyl groups include five- or six-membered heterocyclyl-C1-3-alkyl, such as but not limited to tetrahydropyranylmethyl.

The term “cycloalkylalkyl” used in the present invention refers to an alkyl group substituted by a cycloalkyl moiety as defined above, wherein the alkyl group is defined as per the above definition of alkyl. Examples of cycloalkylalkyl groups include five- or six-membered cycloalkyl-C1-3-alkyl, such as but not limited to cyclopropylmethyl.

The term “substituted alkyl” used in the present invention refers to an alkyl group in which one or more hydrogen atoms are independently replaced by D substituents, wherein the D substituents include, but are not limited to, F, Cl, Br, I, CN, CF₃, OR, R, ═O, ═S, ═NR, ═N+(O)(R), ═N(OR), ═N+(O)(OR), ═N—NRR′, —C(═O)R, —C(═O)OR, —C(═O)NRR′, —NRR′, —N+RR′R″, —N(R)C(═O)R′, —N(R)C(═O)OR′, —N(R)C(═O)NR′R″, —SR, —OC(═O)R, —OC(═O)OR, —OC(═O)NR′R″, —OS(O)₂OR, —OP(═O)(OR)₂, —OP(OR)₂, —P(═O)(OR)₂, —P(═O)(OR)NR′R″, —S(O)R, —S(O)₂R, —S(O)₂NR, —S(O)(OR), —S(O)₂(OR), —SC(═O)R, —SC(═O)OR, ═O, and —SC(═O)NR′R″, wherein each R, R′, and R″ is independently selected from hydrogen, deuterium, alkyl, alkenyl, alkynyl, aryl, and heterocyclyl.

Furthermore, one or more hydrogen atoms in each of the alkenyl, alkynyl, allyl, cycloalkyl, heteroalkyl, heterocyclyl, arylalkyl, heteroarylalkyl, heterocyclylalkyl, cycloalkylalkyl, aryl, or heteroaryl, as defined above, may also be independently substituted by D substituents. The term “halogen” used in the present invention includes fluorine, bromine, chlorine, and iodine.

A bufalin phosphate derivative is a compound having a structure represented by Formula IV and pharmaceutically acceptable salts:

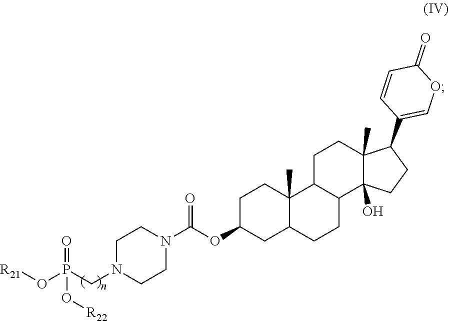

Wherein: R21 and R22 are each independently selected from hydrogen, deuterium, and any one of alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl; n is an integer from 1 to 10.

Preferably, R21 and R22 are each independently selected from hydrogen, deuterium, and any one of the alkyl, cycloalkyl, and aryl groups, and n is an integer from 1 to 3.

Further: The bufalin phosphate derivative is a compound having a structure represented by Formula IVa, IVb, or IVc, and their respective pharmaceutically acceptable salts.

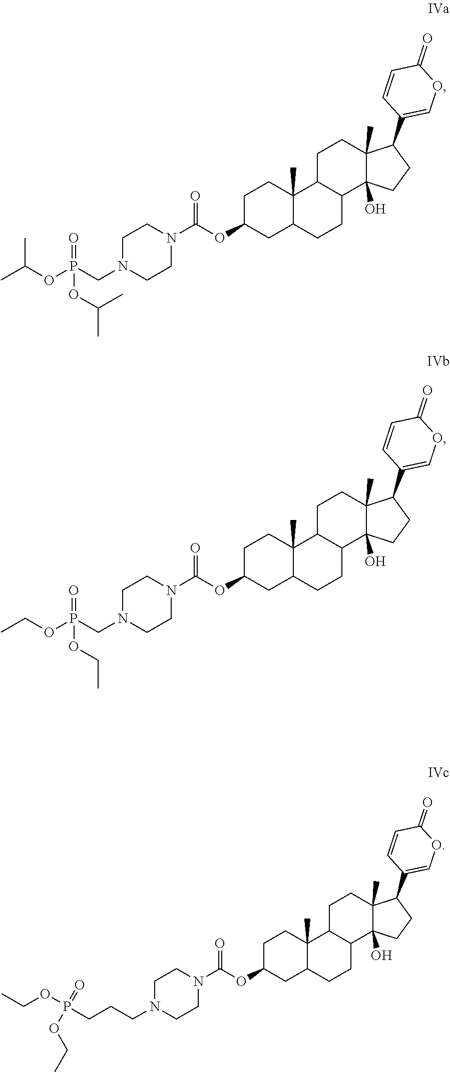 [SMILES: CC(C)OP(=O)(CN1CCN(C(=O)O[C@H]2CC[C@@]3(C)C(CCC4C3CC[C@]3(C)[C@@H](c5ccc(=O)oc5)CC[C@]43O)C2)CC1)OC(C)C ; CCOP(=O)(CN1CCN(C(=O)O[C@H]2CC[C@@]3(C)C(CCC4C3CC[C@]3(C)[C@@H](c5ccc(=O)oc5)CC[C@]43O)C2)CC1)OCC ; CCOP(=O)(CCCN1CCN(C(=O)O[C@H]2CC[C@@]3(C)C(CCC4C3CC[C@]3(C)[C@@H](c5ccc(=O)oc5)CC[C@]43O)C2)CC1)OCC]

Further: The pharmaceutically acceptable salt of the compound is formed by reacting the compound with a pharmaceutically acceptable inorganic acid or organic acid, wherein the inorganic acid is any one of hydrochloric acid, hydrobromic acid, phosphoric acid, nitric acid, or sulfuric acid; and the organic acid is any one of formic acid, acetic acid, propionic acid, succinic acid, 1,5-naphthalenedisulfonic acid, linolenic acid, carbenoxolone, glycyrrhetic acid, oleanolic acid, crataegic acid, ursolic acid, corosolic acid, betulinic acid, boswellic acid, oxalic acid, tartaric acid, lactic acid, salicylic acid, benzoic acid, pentanoic acid, diethylacetic acid, malonic acid, succinic acid, fumaric acid, pimelic acid, adipic acid, maleic acid, malic acid, aminosulfonic acid, phenylpropionic acid, gluconic acid, ascorbic acid, nicotinic acid, isonicotinic acid, methanesulfonic acid, ethanesulfonic acid, p-toluenesulfonic acid, citric acid, or an amino acid.

Secondly, the present invention also provides a pharmaceutical composition of a bufalin phosphate derivative.

A pharmaceutical composition of a bufalin phosphate derivative, comprising a therapeutically effective amount of the aforementioned bufalin phosphate derivative, and one or more of pharmaceutically acceptable carriers, excipients, and adjuvants.

Furthermore, the present invention also provides a bufalin phosphate derivative formulation.

A bufalin phosphate derivative formulation, which is at least one of an injection, a powdered injection, an injection emulsion, a tablet, a pill, a capsule, a paste, a cream, a patch, a liniment, a powder, a spray, an implant, a drop, a suppository, an ointment, or a nano-formulation prepared using the aforementioned pharmaceutical composition, wherein the injection is at least one of a small volume injection, a medium volume injection, or a large volume injection, and the nano-formulation is a liposome.

Additionally, the present invention also provides an application of a bufalin phosphate derivative.

An application of a bufalin phosphate derivative involves using a therapeutically effective amount of the aforementioned bufalin phosphate derivative for preparing at least one of an anti-tumor drug, a cardiovascular and cerebrovascular disease treatment drug, or a nervous system disease treatment drug.

Further: The anti-tumor drug is a drug for treating at least one malignant tumor growing in the esophagus, stomach, intestine, rectum, mouth, pharynx, larynx, lungs, colon, breast, uterus, endometrium, ovaries, prostate, testes, bladder, kidneys, liver, pancreas, bones, connective tissue, skin, eyes, brain, and central nervous system of humans or animals, as well as for treating at least one of thyroid cancer, leukemia, Hodgkin's disease, lymphoma, and myeloma.

Compared with the existing technology, the beneficial effects and significant advancements of the present invention lie in:
1. The bufalin derivatives provided by the present invention are new bufalin series derivatives and their pharmaceutically acceptable salts obtained by introducing modified and different phosphate ester groups on the basis of existing bufalin. Experimental evidence shows that the bufalin derivatives provided by the present invention not only exhibit excellent anti-tumor activity and lower hERG potassium channel inhibitory activity compared to existing bufalin and its derivatives, but also have reduced toxic and side effects. Therefore, they can be used to prepare a variety of anti-tumor drugs, cardiovascular and cerebrovascular disease treatment drugs, and nervous system disease treatment drugs.
2. The bufalin derivatives provided by the present invention possess multiple selectable structural forms and their pharmaceutically acceptable salts. Therefore, they can meet the needs of preparing various pharmaceutical compositions, pharmaceutical formulations, etc., providing a foundation and ideas for the preparation of novel pharmaceutical formulations with better therapeutic effects and lower toxic and side effects. This has broad application prospects.
3. The bufalin derivatives provided by the present invention are characterized by simple and convenient preparation methods, with inexpensive and easily obtainable raw materials, as well as diverse options, facilitating industrial production.
4. The bufalin phosphate derivatives provided by the present invention are new bufalin phosphate series derivatives and their pharmaceutically acceptable salts obtained by introducing modified and different phosphate ester groups on the basis of existing bufalin. Experimental evidence demonstrates that the bufalin phosphate derivatives and their pharmaceutically acceptable salts provided by the present invention exhibit excellent anti-tumor activity. Therefore, they can be used to prepare a variety of anti-tumor drugs, cardiovascular and cerebrovascular disease treatment drugs, and nervous system disease treatment drugs.
5. The bufalin phosphate derivatives provided by the present invention possess multiple selectable structural forms and their pharmaceutically acceptable salts. Therefore, they can meet the needs of preparing various pharmaceutical compositions, pharmaceutical formulations, etc., providing a foundation and ideas for the preparation of novel pharmaceutical formulations with better therapeutic effects and lower toxic and side effects. This has broad application prospects.

### BRIEF DESCRIPTION OF THE DRAWINGS

FIG. 1 is a bar chart showing the inhibitory effect of the bufalin derivative with the structural formula of 1a prepared in the examples of the present invention on the migration of 95D cells at different concentrations;

FIG. 2 is a bar chart showing the inhibitory effect of the bufalin derivative with the structural formula of 1b prepared in the examples of the present invention on the migration of 95D cells at different concentrations;

In the drawings: the results of the cell scratch test demonstrate that the bufalin derivatives with the structural formulas of 1a or 1b prepared in the examples of the present invention exhibit excellent anti-tumor migration effects on 95D cells at different concentrations.

### DETAILED DESCRIPTION OF THE INVENTION

To make the purpose, technical solution, beneficial effects, and significant advancements of the examples of the present invention clearer, the technical solution in the examples of the present invention will be described clearly and completely below in conjunction with the reaction schemes provided in the examples of the present invention. It is obvious that all of these described examples are merely some examples of the present invention, rather than all of them; based on the examples in the present invention, all other examples obtained by ordinary technicians in the art without making creative efforts fall within the scope of protection of the present invention.

It should be noted that the terms “first,” “second,” etc., in the specification and claims of the present invention, as well as in the accompanying drawings of the examples of the present invention, are merely used to distinguish between different objects and are not used to describe a specific order. Furthermore, the term “include” and any variations of it are intended to cover non-exclusive inclusion, for example, a process, method, system, product, or device that includes a series of steps or units is not limited to the listed steps or units, but optionally also includes steps or units that are not listed, or optionally also includes other steps or units that are inherent to these processes, methods, products, or devices.

It should be understood that in the description of the examples of the present invention, some commonly used basic operational terms in the art, such as “heating,” “stirring,” “mixing,” “dissolving,” “washing with water,” “filtering,” “drying,” and so on, should be broadly interpreted. These terms can refer to conventional operations performed using various conventional equipment and instruments in the field, or they can refer to operations performed using the latest equipment, such as programmed operations or unmanned automatic operations. Unless otherwise specifically defined, ordinary technicians in the art can understand the specific meanings of these terms in the context of the present invention based on specific circumstances and adopt specific operational methods to achieve their operational objectives.

It should also be noted that the following specific examples can be combined with each other, and for concepts or processes that are identical or similar, they may not be repeated in some examples. Furthermore, the raw materials, auxiliary materials, reaction equipment, and facilities mentioned in the following specific examples are either commercially available or can be prepared according to existing techniques.

Below, the technical solution of the present invention will be described in detail with specific examples.

### Example 1

This example provides a method for preparing a bufalin derivative with the structure of Formula I.
The method for preparing the bufalin derivative provided in this example is simple and convenient, with inexpensive and easily available raw materials and diverse selection options, making it suitable for industrial production.
To further facilitate understanding of the technical solution for preparing the bufalin derivative provided in Example 1, as well as the specific operational process and achievable results, the following provides further explanation of the preparation method through a specific case as described below.

### Case 1: Preparation of Compound 1a

### Preparation Route:

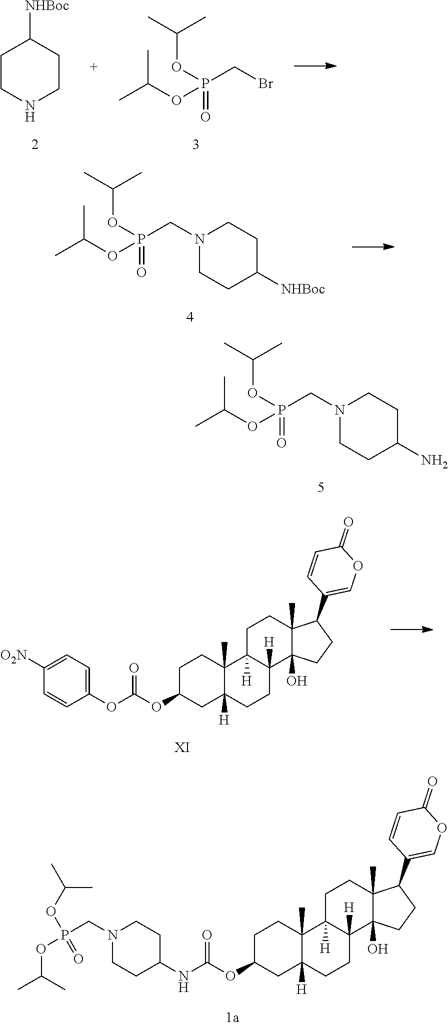

The specific operational process is as follows: 500 mg (2.5 mmol) of compound 2, 647 mg (2.5 mmol) of compound 3, and 266.5 mg (2.5 mmol) of potassium carbonate were added to a flask. Then, 5 mL of acetonitrile was added, and the mixture was heated to 80° C. for a reaction of 12 hours. After that, the mixture was filtered, and the solvent was removed under reduced pressure. The residue was purified by column chromatography to obtain 573 mg of compound 4 as a colorless liquid, with a molar yield of 60.6% relative to compound 2.

Next, 550 mg (1.455 mmol) of compound 4 and 5 mL of trifluoroacetic acid were dissolved together in 5 mL of dichloromethane and reacted at room temperature for 1 hour. After that, the solvent was removed under reduced pressure to obtain compound 5, which was directly used in the next step of the reaction.

Subsequently, 198 mg (0.359 mmol) of (3S,5R,8R,9S,10S,11S,13R,14S,17R)-11,14-dihydro-10,13-dimethyl-12-oxo-17-(2-oxo-2H-pyran-5-yl)hexadecahydro-1H-cyclopenta[a]phenanthren-3-yl-(4-nitrophenyl) carbonate, namely compound XI, obtained by the preparation method disclosed in patent application CN110483608A, was dissolved in 3 mL of dichloromethane. Then, 72 mg (0.718 mmol) of triethylamine and 200 mg (0.718 mmol) of compound 5 were added, and the mixture was reacted at room temperature for 2 hours. After completion of the reaction, the mixture was first washed with saturated saline solution, then dried with anhydrous sodium sulfate, filtered, and the solvent was removed under reduced pressure. The residue was purified by liquid chromatography to obtain 63 mg of compound 1a, with a molar yield of 25.5% relative to compound XI. Test results:

¹H NMR (400 MHz, CDCl₃) δ7.84 (dd, J=9.6, 2.4 Hz, 1H), 7.23 (d, J=2.0 Hz, 1H), 6.26 (d, J=9.6 Hz, 1H), 4.98 (s, 1H), 4.77-4.69 (m, 2H), 4.55 (d, J=6.8 Hz, 1H), 3.48 (s, 1H), 3.01 (d, J=12.0 Hz, 2H), 2.72 (d, J=11.6 Hz, 2H), 2.46 (dd, J=9.2, 6.4 Hz, 1H), 2.32 (t, J=11.2 Hz, 2H), 2.23-2.15 (m, 1H), 2.09-2.02 (m, 1H), 1.93-1.84 (m, 4H), 1.78-1.69 (m, 3H), 1.33 (s, 6H), 1.31 (s, 6H), 0.94 (s, 3H), 0.70 (s, 3H); LC-MS: m z 691.4[M+H]⁺.

### Case 2: Preparation of Compound 1b

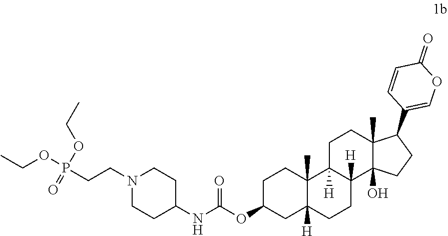

The preparation route and specific operational process for compound 1b are basically the same as those for compound 1a in Case 1, with the only difference being the substitution of diethyl (2-(4-aminopiperidin-1-yl)ethyl)phosphonate for compound 5 in Case 1. This substitution results in the obtainment of compound 1b as a white solid, with a molar yield of 26.0% relative to compound XI. Test results:

¹H NMR (600 MHz, Chloroform-d) δ7.84 (dd, J=9.8, 2.6 Hz, 1H), 7.23 (dd, J=2.6, 1.1 Hz, 1H), 6.26 (dd, J=9.8, 1.0 Hz, 1H), 4.98 (s, 1H), 4.58 (d, J=8.0 Hz, 1H), 4.18-3.98 (m, 4H), 3.52 (s, 1H), 2.86 (s, 2H), 2.68 (q, J=8.3 Hz, 2H), 2.46 (dd, J=9.7, 6.5 Hz, 1H), 2.23-2.11 (m, 3H), 2.10-1.93 (m, 6H), 1.87 (d, J=14.1 Hz, 5H), 1.78-1.62 (m, 6H), 1.61-1.44 (m, 8H), 1.42-1.37 (m, 1H), 1.32 (t, J=7.1 Hz, 7H), 1.30-1.23 (m, 4H), 0.95 (s, 3H), 0.70 (s, 3H); HRMS (ESI, positive) m/z calcd for C₃₆H₅₇N₂O₈P[M+H]⁺: 677.3931, found: 677.3925.

### Case 3: Preparation of Compound 1c

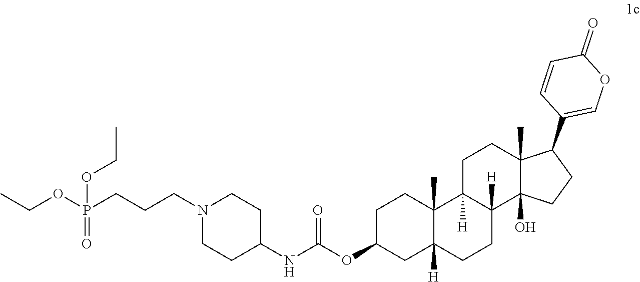

The preparation route and specific operational process for compound 1c are essentially the same as those for compound 1a in Case 1, with the sole difference being the substitution of diethyl (3-(4-aminopiperidin-1-yl)propyl)phosphonate for compound 5 in Case 1. This substitution leads to the obtainment of compound 1c as a white solid, with a molar yield of 56.6% relative to compound XI. Test results:

¹H NMR (600 MHz, Chloroform-d) δ7.83 (dd, J=9.7, 2.6 Hz, 1H), 7.22 (d, J=2.6, 1.1 Hz, 1H), 6.25 (d, J=9.6, 1.0 Hz, 1H), 4.97 (s, 1H), 4.66-4.56 (m, 1H), 4.16-3.99 (m, 4H), 3.53 (s, 1H), 2.91 (s, 2H), 2.52-2.41 (m, 3H), 2.23-2.13 (m, 3H), 2.08-2.01 (m, 1H), 1.96 (d, J=12.8 Hz, 2H), 1.91-1.79 (m, 4H), 1.78-1.70 (m, 5H), 1.69-1.61 (m, 3H), 1.53-1.44 (m, 4H), 1.43-1.35 (m, 2H), 1.31 (t, J=7.0 Hz, 9H), 1.28-1.22 (m, 4H), 1.22-1.14 (m, 1H), 0.94 (s, 3H), 0.69 (s, 3H); HRMS (ESI, positive) m/z calcd for C₃₇H₅₉N₂O₈P[M+H]⁺: 691.4087, found: 691.4082.

### Case 4: Preparation of Compound 1d

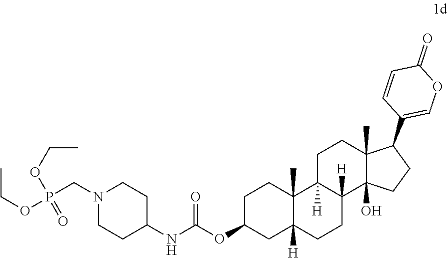

The preparation route and specific operational process for compound 1d are largely the same as those for compound 1a in Case 1, with the only difference being the substitution of diethyl ((4-aminopiperidin-1-yl)methyl)phosphonate for compound 5 in Case 1. This substitution results in the obtainment of compound 1d as a white solid, with a molar yield of 75.8% relative to compound XI. Test results:

¹H NMR (600 MHz, Chloroform-d) δ7.84 (dd, J=9.8, 2.6 Hz, 1H), 7.25-7.19 (m, 1H), 6.27 (d, J=9.7 Hz, 1H), 4.98 (d, J=4.1 Hz, 1H), 4.56 (s, 1H), 4.28-4.02 (m, 3H), 3.51 (s, 1H), 3.04 (s, 1H), 2.82 (s, 1H), 2.47 (dd, J=9.8, 6.6 Hz, 1H), 2.39 (s, 1H), 2.24-2.15 (m, 1H), 2.10-2.01 (m, 1H), 1.94 (s, 1H), 1.87 (t, J=14.3 Hz, 2H), 1.73 (d, J=3.3 Hz, 2H), 1.69 (d, J=3.8 Hz, 1H), 1.64 (s, 6H), 1.60 (s, 1H), 1.53 (s, 1H), 1.51 (s, 1H), 1.49 (s, 1H), 1.42 (d, J=4.4 Hz, 1H), 1.37 (d, J=3.7 Hz, 1H), 1.34 (t, J=7.1 Hz, 6H), 1.31-1.28 (m, 3H), 1.27-1.24 (m, 4H), 1.19 (s, 2H), 0.95 (s, 2H), 0.88 (t, J=6.9 Hz, 1H), 0.84 (s, 1H), 0.70 (s, 2H); HRMS (ESI, positive) m/z calcd for C₃₅H₅₅N₂O₈P[M+H]⁺: 663.3774, found: 663.3769.

### Case 5: Preparation of Compound 1e

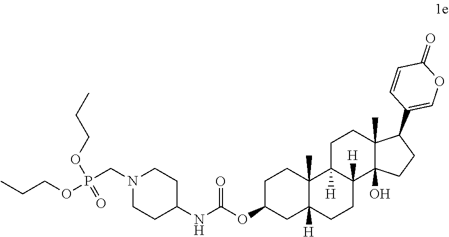

The preparation route and specific operational process for compound 1e are essentially the same as those for compound 1a in Case 1, with the sole difference being the substitution of dipropyl ((4-aminopiperidin-1-yl)methyl)phosphonate for compound 5 in Case 1. This substitution leads to the obtainment of compound 1e as a white solid, with a molar yield of 52.2% relative to compound XI. Test results:

¹H NMR (600 MHz, Chloroform-d) δ7.83 (dd, J=9.8, 2.6 Hz, 1H), 7.22 (dd, J=2.7, 1.1 Hz, 1H), 6.25 (dd, J=9.8, 1.1 Hz, 1H), 4.97 (s, 1H), 4.57 (s, 1H), 4.10-3.94 (m, 4H), 3.50 (s, 1H), 3.03 (d, J=11.3 Hz, 2H), 2.82 (d, J=11.5 Hz, 2H), 2.49-2.43 (m, 1H), 2.39 (s, 1H), 2.25-2.14 (m, 1H), 2.09-2.00 (m, 1H), 1.93 (d, J=12.6 Hz, 2H), 1.89-1.83 (m, 2H), 1.76-1.61 (m, 10H), 1.56-1.44 (m, 7H), 1.43-1.37 (m, 1H), 1.37-1.31 (m, 2H), 1.30-1.24 (m, 4H), 1.21-1.13 (m, 1H), 0.98-0.91 (m, 9H), 0.69 (s, 3H); HRMS (ESI, positive) m/z calcd for C₃₇H₅₉N₂O₈P[M+H]⁺: 691.4087, found: 691.4082.

### Case 6: Preparation of Compound 1f

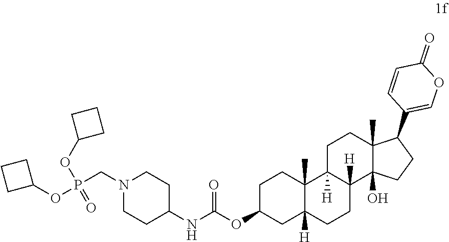

The preparation route and specific operational process for compound 1f are largely the same as those for compound 1a in Case 1, with the only difference being the substitution of dicyclobutyl ((4-aminopiperidin-1-yl)methyl)phosphonate for compound 5 in Case 1. This substitution results in the obtainment of compound 1f as a white solid, with a molar yield of 61.7% relative to compound XI. Test results:

¹H NMR (600 MHz, Chloroform-d) δ7.83 (dd, J=9.8, 2.6 Hz, 1H), 7.22 (d, J=2.6, 1.1 Hz, 1H), 6.25 (d, J=9.7, 1.1 Hz, 1H), 4.97 (s, 1H), 4.84-4.70 (m, 2H), 4.56 (s, 1H), 3.49 (s, 1H), 2.99 (d, J=11.4 Hz, 2H), 2.80-2.68 (m, 2H), 2.48-2.41 (m, 1H), 2.36-2.27 (m, 5H), 2.22-2.10 (m, 5H), 2.09-2.01 (m, 1H), 1.93-1.83 (m, 4H), 1.78-1.68 (m, 5H), 1.67-1.61 (m, 2H), 1.54-1.44 (m, 8H), 1.42-1.35 (m, 2H), 1.34-1.22 (m, 7H), 1.21-1.15 (m, 1H), 0.93 (s, 3H), 0.69 (s, 3H); HRMS (ESI, positive) m/z calcd for C₃₉H₅₉N₂O₈P[M+H]⁺: 715.4087, found: 715.4082.

### Case 7: Preparation of Compound 1g

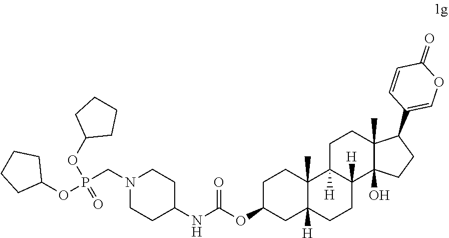

The preparation route and specific operational process for compound 1g are essentially the same as those for compound 1a in Case 1, with the sole difference being the substitution of dicyclopentyl ((4-aminopiperidin-1-yl)methyl)phosphonate for compound 5 in Case 1. This substitution leads to the obtainment of compound 1g as a white solid, with a molar yield of 58.0% relative to compound XI. Test results:

¹H NMR (600 MHz, Chloroform-d) δ7.83 (dd, J=9.7, 2.6 Hz, 1H), 7.22 (dd, J=2.6, 1.1 Hz, 1H), 6.25 (dd, J=9.8, 1.1 Hz, 1H), 5.06-4.86 (m, 3H), 4.56 (d, J=8.0 Hz, 1H), 3.48 (s, 1H), 3.00 (d, J=11.2 Hz, 2H), 2.73 (d, J=11.4 Hz, 2H), 2.50-2.29 (m, 3H), 2.25-2.12 (m, 1H), 2.10-2.00 (m, 1H), 1.95-1.78 (m, 13H), 1.77-1.66 (m, 8H), 1.65-1.59 (m, 3H), 1.54-1.44 (m, 6H), 1.42-1.12 (m, 10H), 0.93 (s, 3H), 0.69 (s, 3H); HRMS (ESI, positive) m/z calcd for C₄₁H₆₃N₂O₈P[M+H]⁺: 743.4400, found: 743.4395.

### Case 8: Preparation of Compound 1h

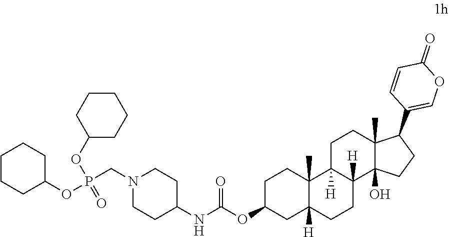

The preparation route and specific operational process for compound 1h are largely the same as those for compound 1a in Case 1, with the only difference being the substitution of dicyclohexyl ((4-aminopiperidin-1-yl)methyl)phosphonate for compound 5 in Case 1. This substitution results in the obtainment of compound 1h as a white solid, with a molar yield of 61.1% relative to compound XI. Test results:

¹H NMR (600 MHz, Chloroform-d) δ7.83 (dd, J=9.8, 2.6 Hz, 1H), 7.22 (dd, J=2.6, 1.1 Hz, 1H), 6.25 (d, J=9.7 Hz, 1H), 4.97 (s, 1H), 4.56 (d, J=8.0 Hz, 1H), 4.49-4.38 (m, 2H), 3.48 (s, 1H), 3.01 (d, J=11.3 Hz, 2H), 2.75 (d, J=11.5 Hz, 2H), 2.47-2.42 (m, 1H), 2.35 (s, 2H), 2.23-2.12 (m, 1H), 2.10-1.98 (m, 1H), 1.95-1.82 (m, 8H), 1.77-1.67 (m, 7H), 1.67-1.60 (m, 2H), 1.58 (dd, J=11.8, 3.2 Hz, 1H), 1.55-1.42 (m, 13H), 1.37-1.15 (m, 13H), 0.93 (s, 3H), 0.69 (s, 3H); HRMS (ESI, positive) m/z calcd for C₄₃H₆₇N₂O₈P[M+H]⁺: 771.4713, found: 771.4708.

### Case 9: Preparation of Compound 1i

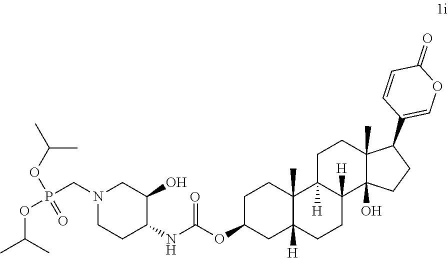

The preparation route and specific operational process for compound 1i are essentially the same as those for compound 1a in Case 1, with the sole difference being the substitution of diisopropyl (((3R,4R)-4-amino-3-hydroxypiperidin-1-yl)methyl)phosphonate for compound 5 in Case 1. This substitution leads to the obtainment of compound 1i as a white solid, with a molar yield of 64.6% relative to compound XI. Test results:

¹H NMR (600 MHz, Chloroform-d) δ7.87-7.82 (m, 1H), 7.24 (dd, J=2.6, 1.2 Hz, 1H), 6.28 (d, J=9.7 Hz, 1H), 5.01 (s, 1H), 4.84 (s, 1H), 4.79-4.71 (m, 1H), 3.63 (s, 1H), 3.50 (s, 1H), 3.41 (s, 1H), 3.23 (s, 1H), 3.14 (s, 1H) 2.83 (d, J=11.8 Hz, 1H), 2.50-2.45 (m, 1H), 2.21 (dt, J=12.9, 9.3 Hz, 1H) 2.11-2.03 (m, 1H), 2.01 (s, 1H), 1.93-1.86 (m, 2H), 1.77-1.67 (m, 4H) 1.67-1.56 (m, 5H), 1.50 (s, 1H), 1.46-1.37 (m, 2H), 1.34 (dd, J=6.2, 1.3 Hz, 12H), 1.28 (d, J=17.7 Hz, 3H), 1.24-1.19 (m, 2H), 0.96 (s, 3H), 0.71 (s, 3H); HRMS (ESI, positive) m/z calcd for C₃₇H₅₉N₂O₉P[M+H]⁺: 707.4036, found: 707.4031.

### Case 10: Preparation of Compound 1j

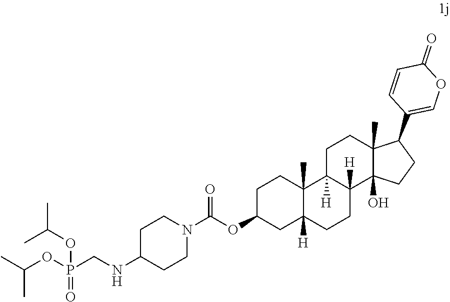

The preparation route and specific operational process for compound 1j are largely the same as those for compound 1a in Case 1, with the only difference being the substitution of diisopropyl ((piperidin-4-ylamino)methyl)phosphonate for compound 5 in Case 1. This substitution results in the obtainment of compound 1j as a white solid, with a molar yield of 59.5% relative to compound XI. Test results:

¹H NMR (600 MHz, Chloroform-d) δ7.83 (dd, J=9.8, 2.6 Hz, 1H), 7.20 (dd, J=2.6, 1.1 Hz, 1H), 6.22 (dd, J=9.8, 1.0 Hz, 1H), 4.97 (s, 1H), 4.75-4.65 (m, 2H), 4.00 (s, 2H), 2.95-2.80 (m, 4H), 2.71-2.62 (m, 1H), 2.45-2.40 (m, 1H), 2.20-2.12 (m, 1H), 2.06-1.99 (m, 1H), 1.89-1.79 (m, 5H), 1.75-1.66 (m, 3H), 1.64-1.57 (m, 3H), 1.56-1.50 (m, 3H), 1.49-1.43 (m, 2H) 1.42-1.33 (m, 2H), 1.31-1.22 (m, 18H), 1.20-1.11 (m, 1H), 0.92 (s, 3H) 0.67 (s, 3H); HRMS (ESI, positive) m/z calcd for C₃₇H₅₉N₂O₈P[M+H]⁺: 691.4087, found: 691.4082.

### Case 1l: Preparation of Compound 1k

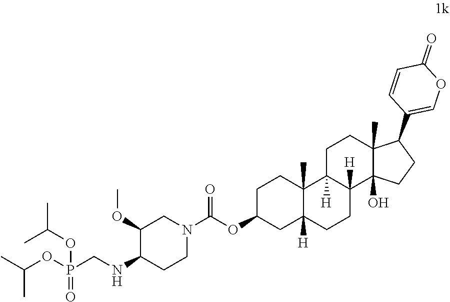

The preparation route and specific operational process for compound 1k are essentially the same as those for compound 1a in Case 1, with the sole difference being the substitution of diisopropyl (((((3S,4R)-3-methoxypiperidin-4-yl)amino)methyl)phosphonate for compound 5 in Case 1. This substitution leads to the obtainment of compound 1k as a white solid, with a molar yield of 73.9% relative to compound XI. Test results:

¹H NMR (600 MHz, Chloroform-d) δ7.85 (dd, J=9.7, 2.6 Hz, 1H), 7.24 (dd, J=2.6, 1.1 Hz, 1H), 6.28 (dd, J=9.7, 1.1 Hz, 1H), 5.02 (s, 1H), 4.78 (dp, J=12.6, 6.3, 5.6 Hz, 2H), 3.50 (s, 1H), 3.42 (s, 3H), 3.02 (s, 4H), 2.48 (dd, J=9.7, 6.5 Hz, 1H), 2.21 (dt, J=12.7, 9.4 Hz, 1H), 2.12-2.04 (m, 1H), 1.89 (ddd, J=14.4, 10.1, 4.8 Hz, 2H), 1.82-1.68 (m, 7H), 1.64-1.49 (m, 8H), 1.47-1.40 (m, 2H), 1.38-1.35 (m, 12H), 1.34-1.25 (m, 6H), 1.23-1.18 (m, 2H), 0.96 (s, 3H), 0.72 (s, 3H); HRMS (ESI, positive) m/z calcd for C₃₈H₆₁N₂O₉P[M+H]⁺: 721.4193, found: 721.4187.

### Case 12: Preparation of Compound 1l

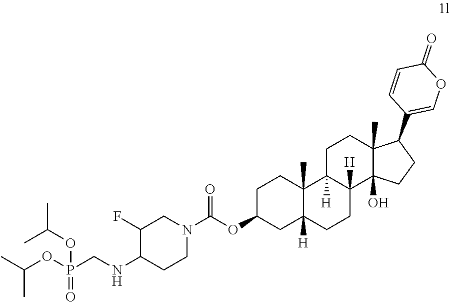

The preparation route and specific operational process for compound 1l are largely the same as those for compound 1a in Case 1, with the only difference being the substitution of diisopropyl (((3-fluoropiperidin-4-yl)amino)methyl)phosphonate for compound 5 in Case 1. This substitution results in the obtainment of compound 1l as a white solid, with a molar yield of 75.8% relative to compound XI. Test results:

¹H NMR (600 MHz, Chloroform-d) δ7.85 (dd, J=9.7, 2.5 Hz, 1H), 7.24 (dd, J=2.5, 1.0 Hz, 1H), 6.27 (dd, J=9.8, 1.1 Hz, 1H), 5.01 (s, 1H), 4.75 (dt, J=12.6, 6.3 Hz, 2H), 3.05 (t, J=15.5 Hz, 2H), 2.95 (s, 2H), 2.47 (dd, J=9.8, 6.5 Hz, 1H), 2.20 (dt, J=12.6, 9.4 Hz, 1H), 2.09-1.99 (m, 2H), 1.73 (d, J=19.0 Hz, 10H), 1.59-1.54 (m, 9H), 1.51 (d, J=13.7 Hz, 5H), 1.35 (dd, J=6.2, 0.8 Hz, 12H), 1.33-1.28 (m, 4H), 1.26 (s, 3H), 1.20 (d, J=12.9 Hz, 1H), 0.96 (d, J=1.8 Hz, 3H), 0.70 (s, 3H); HRMS (ESI, positive) m/z calcd for C₃₇H₅₈FN₂O₈P[M+H]⁺: 709.3993, found: 709.3988.

From the descriptions of Cases 1 to 12 above, it can be seen that: The preparation method for bufalin derivatives provided in this example is convenient and straightforward, with inexpensive and easily obtainable raw materials. It offers a diverse selection and is conducive to industrial production, featuring high yields and potential for significant economic benefits.

### Example 2

This example provides a method for preparing a pharmaceutically acceptable salt of a bufalin derivative.

The bufalin derivatives having the structures represented by Formula I, Formula II, or Formula III obtained in Example 1 above, or more specifically, the 1a to 1l bufalin derivatives prepared in the various cases of Example 1, are each reacted with a pharmaceutically acceptable inorganic acid or organic acid to obtain the corresponding salt of each bufalin derivative.

Since the relevant reactions are basic and commonly used in this field, for brevity, this example does not provide a detailed description.

In the process of preparing the pharmaceutically acceptable salt of the bufalin derivative mentioned above: The inorganic acid can be any one of hydrochloric acid, hydrobromic acid, phosphoric acid, nitric acid, or sulfuric acid; the organic acid can be any one of formic acid, acetic acid, propionic acid, succinic acid, 1,5-naphthalenedisulfonic acid, linoleic acid, carbenoxolone, glycyrrhetic acid, oleanolic acid, crataegic acid, ursolic acid, corosolic acid, betulinic acid, frankincense acid, oxalic acid, tartaric acid, lactic acid, salicylic acid, benzoic acid, valeric acid, diethylacetic acid, malonic acid, succinic acid (note: succinic acid is repeated here and should be considered as one option), fumaric acid, pimelic acid, adipic acid, maleic acid, malic acid, sulfamic acid, phenylpropionic acid, gluconic acid, ascorbic acid, nicotinic acid, isonicotinic acid, methanesulfonic acid, ethanesulfonic acid, p-toluenesulfonic acid, citric acid, or an amino acid.

### Example 3

This example provides a method for preparing a pharmaceutical composition of a bufalin derivative.

A therapeutically effective amount of the bufalin derivative having the structure represented by Formula I, Formula II, or Formula III obtained in Example 1 above, or the pharmaceutically acceptable salt of the bufalin derivative having the structure represented by Formula I, Formula II, or Formula III obtained in Example 2 above, or more specifically, a therapeutically effective amount of the 1a to 1l bufalin derivatives prepared in the various cases of Example 1 or the pharmaceutically acceptable salts of the 1a to 1l bufalin derivatives prepared in Example 2, is mixed or blended with one or more pharmaceutically acceptable carriers, excipients, and adjuvants to obtain the corresponding pharmaceutical composition of each bufalin derivative or its pharmaceutically acceptable salt.

Since the relevant operations are basic and commonly used in this field, for brevity, this example does not provide a detailed description.

### Example 4

This example provides a method for preparing a pharmaceutical formulation of a bufalin derivative.

The pharmaceutical composition of each bufalin derivative or its pharmaceutically acceptable salt obtained in Example 3 is processed according to the preparation methods for existing pharmaceutical formulations to produce at least one type of pharmaceutical formulation, including but not limited to injections, powdered injections, emulsions for injection, tablets, pills, capsules, pastes, creams, patches, liniments, powders, sprays, implants, drops, suppositories, ointments, or nano-formulations. Among them, the injections can be at least one type of small-volume injection, medium-volume injection, or large-volume injection, and the nano-formulations can be liposomes.

Since the relevant operations are basic and commonly used in this field, for brevity, this example does not provide a detailed description.

### Effect Example 1

This effect example is used to characterize the in vitro antitumor activity of the bufalin derivatives 1a to 1l prepared in Example 1.

### Test Method:

The CCK-8 method described in “Methods for New Drug Pharmacological Research” (edited by Lv Qiujun, 2007: 242-243) was adopted.

The cell lines used were human lung adenocarcinoma cell line A549 and human colorectal cancer cell line HCT116, which were cryopreserved and passaged in the pharmacological laboratory of the Shanghai Institute of Pharmaceutical Industry. The culture medium was DMEM supplemented with 10% FBS and antibiotics.

### In Vitro Activity Testing Procedure:

Cells in logarithmic growth phase were seeded into 96-well plates at a density of 7×10³ cells per well and incubated at 37° C. in a 5% CO₂ environment for 24 hours to maintain a normal physiological pH.

After cell attachment, different concentrations of the test compounds, namely the bufalin derivatives 1a to 1l prepared in Example 1, as well as the compound doxorubicin, were added to triplicate wells, with 0.1% DMSO added as a control.

After 72 hours, 10 μL of CCK-8 cell counting kit was added to each well, and the plates were incubated again at 37° C. for 0.5 to 1 hour. The absorbance (OD) was read at 450 nm on a full-featured microplate reader (Biotek Synergy H2), and the concentration causing 50% cell growth inhibition (IC₅₀) was calculated by simulation using GraphPad Prism software. The test results are shown in Table 1.

TABLE 1
In Vitro Antitumor Activity of the Bufalin Derivatives 1a to 1l
Prepared in Example 1

| | IC₅₀ (μM) | |
|---|---|---|
| compound | A549 | HCT116 |
| 1a | 0.020 | 0.043 |
| 1b | <0.004 | <0.004 |
| 1c | <0.004 | <0.004 |
| 1d | 0.037 | 0.024 |
| 1e | 0.058 | 0.022 |
| 1f | 0.026 | 0.067 |
| / | / | / |
| 1g | 0.422 | 0.289 |
| 1h | 0.023 | 0.336 |
| 1i | 0.015 | 0.008 |
| 1j | 0.216 | 0.020 |
| 1k | 0.239 | 0.143 |
| 1l | 0.283 | 0.120 |
| Doxorubicin | 0.920 | 0.054 |

### The Above Experimental Results Indicate:

The bufalin derivatives 1a to 1l provided in Example 1 exhibit excellent antitumor activity and demonstrate superior activity against lung cancer and colorectal cancer. Their activity against A549 is superior to that of the positive control drug doxorubicin, and among them, seven bufalin derivatives also show better activity against HCT116 than doxorubicin.

Although Table 1 only lists the in vitro antitumor activity test data for the bufalin derivatives 1a to 1l provided in the efficacy example corresponding to Example 1, based on related and similar experiments, it can be concluded that the bufalin derivatives with the structures represented by Formula I, Formula II, or Formula III provided in Example 1 of this invention, as well as the pharmaceutically acceptable salts of these bufalin derivatives with the same structures provided in Example 2, all possess antitumor activities similar to those of the aforementioned bufalin derivatives 1a to 1l. They demonstrate excellent activity against lung cancer and colorectal cancer, with superior activity against A549 compared to the positive control drug doxorubicin. Furthermore, some of them also show better activity against HCT116 than doxorubicin. Based on these findings, it can be considered that the bufalin derivatives and their salts provided by this invention can be used for preparing antitumor medications.

### Efficacy Example 2

This efficacy example is used to characterize the in vivo antitumor activity of the bufalin derivative 1a prepared in Example 1 against HCT116 colorectal cancer cells.

Experimental Method: The test animals were male SPF-grade BALB/C nude mice weighing 18 to 20 grams.

Experimental Procedure: Well-grown HCT116 colorectal cancer tumor masses were cut into uniform small pieces of approximately 3 mm in size under aseptic conditions. A cannula needle was used to inoculate one piece subcutaneously into the right axilla of each test mouse. Nine days after inoculation, the average tumor volume was approximately 113 to 119 mm³. The animals were then regrouped based on tumor size, eliminating those with excessively large or small tumors, to ensure that the average tumor volume was consistent among the experimental groups. Administration by intraperitoneal injection was then initiated according to the following protocol, with a dosing volume of 0.2 mL per 20 grams of body weight:

From the 9th day of inoculation, the maximum tumor length (a, in mm) and the perpendicular maximum tumor width (b, in mm) were measured twice a week using a digital electronic caliper.

The tumor volume (TV) was calculated using the formula: TV=ab²/2.

The relative tumor volume (RTV) was calculated using the formula: RTV=Vt/Vo, where Vo is the tumor volume measured at the time of cage distribution (i.e., day 1) and Vt is the tumor volume at each subsequent measurement.

On the 27th day after inoculation (day 19), the animals were euthanized, weighed, dissected to remove the tumor masses, and the tumor weights were measured. Tumor volume inhibition TGI was determined by the formula of 1−RTVₜᵤₘₒᵣ/RTV_{control}×100%, where RTV is the relative tumor volume.

The experimental results are presented in Table 2.

TABLE 2
In Vivo Antitumor Activity of the Bufalin Derivative 1a Prepared in Example
1 Against HCT116 Colorectal Cancer Cells Xenografts

| | | | | | Animal weight | | |
|---|---|---|---|---|---|---|---|
| | dose | Dosing | Animal | | (After tumor | RTV | TGI |
| entry | (mg/kg) | regimen | Begin | End | removal) (g) | (d18) | (%) |
| Control | 10 ml/kg | | 9 | 9 | 21.73 ± 0.59 | 12.60 ± 1.11 | |
| 5- | 15 | ip × 7 | 6 | 6 | 19.20 ± 1.84 | 5.52 ± 0.81** | 56.16 |
| Fluorouracil | | | | | | | |
| 1a | 10 | ip × 7, qod | 6 | 6 | 20.48 ± 2.13 | 2.24 ± 0.53** | 82.22 |

Comparison with Control Group: **P<0.01.

### The Above Experimental Results Indicate:

The bufalin derivativela prepared in Example 1, when administered by intraperitoneal injection, exhibits excellent in vivo antitumor activity with a tumor inhibition rate of up to 82.22% against HCT116 colorectal cancer cells.

### Efficacy Example 3

This efficacy example is used to characterize the in vivo antitumor activity of the bufalin derivative 1c prepared in Example 1 against HCT116 colorectal cancer cells.

This efficacy example employs the same test animals and experimental methods as Efficacy Example 2, with the only difference being that the average tumor volume was approximately 130 to 140 mm³ on the 13th day after inoculation of HCT116 colorectal cancer tumor masses into the test mice. Subsequently, the animals were regrouped based on tumor size and administered the drug according to the same protocol as in Efficacy Example 2. The animals were euthanized on the 30th day after inoculation (i.e., day 18), and their body weights and tumor weights were measured after dissection. The experimental results are presented in Table 3.

TABLE 3
In Vivo Antitumor Activity of the Bufalin Derivative 1c Prepared in Example
1 Against HCT116 Colorectal Cancer Cells Xenografts

| | | | | | Animal weight | | |
|---|---|---|---|---|---|---|---|
| | dose | Dosing | Animal | | (After tumor | RTV | TGI |
| entry | (mg/kg) | regimen | Begin | End | removal) (g) | (d18) | (%) |
| Control | 10 ml/kg | | 10 | 10 | 20.63 ± 1.40 | 18.08 ± 3.84 | |
| 5- | 15 | ip × 7 | 6 | 6 | 21.84 ± 1.52 | 7.35 ± 1.55** | 59.37 |
| Fluorouracil | | | | | | | |
| 1c | 1 | iv × 7, qod | 6 | 6 | 21.02 ± 3.08 | 6.56 ± 1.79** | 63.69 |

Comparison with Control Group: **P<0.01.

The above experimental results indicate:

The bufalin derivative 1c prepared in Example 1, when administered intravenously, exhibits excellent in vivo antitumor activity with a tumor inhibition rate of 63.69% against HCT116 colorectal cancer cells.

Although Table 2 only presents the experimental data on the in vivo antitumor activity of the bufalin derivative 1a prepared in Example 1 against HCT116 colorectal cancer cells from Efficacy Example 2, and Table 3 only presents the corresponding data for the bufalin derivative 1c from Efficacy Example 3, based on related similar experiments, it can be concluded that other bufalin derivatives with the structures shown in Formula I, II, or III provided in Example 1 of the present invention, or their pharmaceutically acceptable salts with the structures shown in Formula I, II, or III provided in Example 2, all possess antitumor activities similar to those of the aforementioned bufalin derivatives 1a and 1c. Accordingly, it can be considered that the bufalin derivatives and their salts provided by the present invention can be used for preparing antitumor drugs.

### Efficacy Example 4

This efficacy example is used to characterize the effects of the two bufalin derivatives 1a and 1c prepared in Example 1 on the hERG potassium channel.

Experimental Method: CHO-hERG cells were cultured in a 175 cm² flask until the cell density reached 60 to 80%. The culture medium was then removed, and the cells were washed once with 7 mL of Phosphate Buffered Saline (PBS). Subsequently, 3 mL of gentle cell detachment solution (Detachin) was added for digestion. Once the digestion was complete, 7 mL of culture medium was added to neutralize the solution. The cells were then centrifuged, and the supernatant was aspirated. To ensure a cell density of 2 to 5×10⁶/mL, 5 mL of culture medium was added to resuspend the cells.

The entire process of forming single-cell high-impedance seals and whole-cell recording mode was automatically completed by the high-throughput, fully automated patch clamp system, namely the Qpatch instrument. After obtaining the whole-cell recording mode, the cells were clamped at −80 mV. Prior to applying a 5-second+40 mV depolarization stimulus, a 50-millisecond −50 mV prepulse was given, followed by a repolarization to −50 mV for 5 seconds, and then returning to −80 mV This voltage stimulus was applied every 15 seconds. After recording for 2 minutes, the extracellular solution was recorded for 5 minutes, and then the drug administration process began. Each test concentration of the two bufalin derivatives, 1a and 1c, used for testing was administered for 2.5 minutes. The positive control compound, cisapride at 0.3 M, was also tested, with at least 3 cells tested per concentration (i.e., n≥3).

On the day of testing, the bufalin derivatives 1a and 1c for testing were each prepared as 15 mM DMSO stock solutions. These stock solutions were then diluted 500-fold with extracellular solution to obtain the final concentrations required for testing.

### Preparation of the Positive Control Compound, Cisapride:

10 μL of a 150 M cisapride DMSO stock solution was added to 4990 μL of extracellular solution to obtain a final test concentration of 300 nM after 500-fold dilution. The final DMSO content in the test concentration did not exceed 0.2%, a concentration that has no effect on the hERG potassium channel.

The entire dilution process for the bufalin derivatives 1a and 1c used for testing was completed by the Bravo instrument, a protein sample pretreatment platform.

The test results are shown in Table 4.

TABLE 4
Activity Test Data of the Two Bufalin Derivatives 1a and 1c
Prepared in Example 1 on the hERG Potassium Channel

| compound | Test concentration | Inhibition rate |
|---|---|---|
| 1a | 30 μM | 21.3 ± 0.18% |
| 1c | 30 μM | 53.8 ± 4.13% |
| Cisapride | 0.3 μM | 97.4 ± 1.08% |

### The Above Test Results Indicate:

At a concentration of 30 M, the inhibition rates of the two bufalin derivatives 1a and 1c prepared in Example 1 on the hERG potassium channel were 21.3±0.18% and 53.8±4.13%, respectively, which are significantly lower than the inhibition rate of 97.4±1.08% observed with the positive control drug cisapride at 0.3 μM. This suggests that both 1a and 1c bufalin derivatives exhibit low inhibitory activity against the hERG potassium channel and thus have low cardiac toxicity.

Although Table 4 only presents the activity test data of the two bufalin derivatives, 1a and 1c, prepared in Example 1 on the hERG potassium channel from Efficacy Example 4, based on related similar experiments, it can be concluded that other bufalin derivatives with the structures shown in Formula I, II, or III provided in Example 1 of the present invention, or their pharmaceutically acceptable salts with the structures shown in Formula I, II, or III provided in Example 2, all possess similar inhibitory activity against the hERG potassium channel as the aforementioned 1a and 1c bufalin derivatives. Accordingly, it can be considered that the bufalin derivatives and their salts provided by the present invention exhibit low cardiac toxicity and can be used for preparing therapeutic drugs for cardiovascular and cerebrovascular diseases or neurological diseases.

### Efficacy Example 5

This efficacy example is used to characterize the inhibitory effect of the two bufalin derivatives, 1a and 1b, prepared in Example 1 on the migration of 95D cells at different concentrations.

Test Method: Draw three parallel lines with a spacing of approximately 0.5 cm on the outer bottom surface of a 6-well plate. After evenly seeding the tumor cells 95D onto the plate (5×10⁵ cells/well), create a cell scratch using a pipette. Wash the cells three times with phosphate-buffered saline (PBS) to remove the scratched-off cells. Then, add medium that has been pre-mixed with different concentrations of compound 1a or 1b.

Place the plate in a 37° C., 5% CO₂ incubator for culturing. Sample the cells at 0, 12, 24, 36, and 48 hours, and take photographs under a microscope.

Using ImageJ software, measure the area and height of the scratch site. The average scratch width can be obtained by dividing the area by the height. Subtract the scratch width at the end of the experiment from the scratch width at the 0-hour point to obtain the distance of cell migration. Then, use this distance value to calculate the cell migration index (MI) according to the following formula:

Formula:

Cell Migration Index(MI)=(Initial Blank Width−Blank Width at a Specific Time Point)/Initial Blank Width.

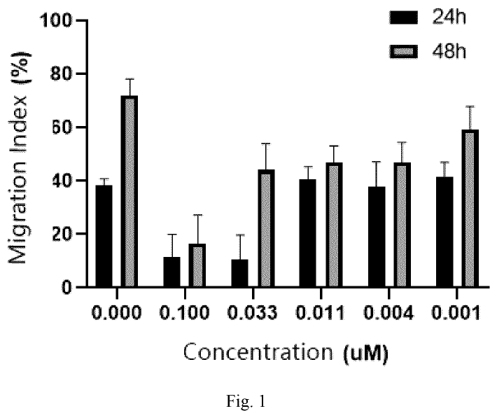

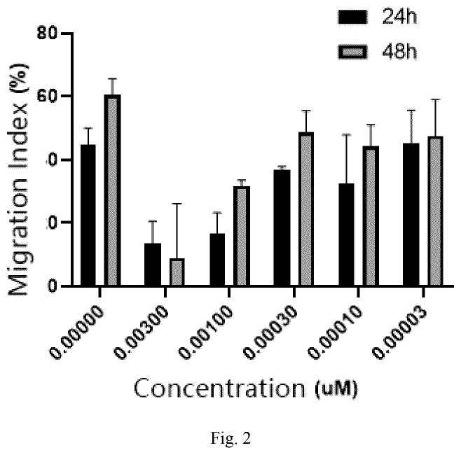

### The Test Results are Shown in FIG. 1 and FIG. 2:

FIG. 1 is a bar chart showing the inhibitory effect of the bufalin derivative with the structural formula of 1a prepared in the examples of the present invention on the migration of 95D cells at different concentrations; and FIG. 2 is a bar chart showing the inhibitory effect of the bufalin derivative with the structural formula of 1b prepared in the examples of the present invention on the migration of 95D cells at different concentrations.

From FIGS. 1 and 2, it can be observed that: The results of the cell scratch test demonstrate that the bufalin derivatives with the structural formulas of 1a or 1b prepared in the examples of the present invention exhibit excellent anti-tumor migration effects on 95D cells at different concentrations. Therefore, they can be used for preparing drugs that inhibit tumor metastasis.

In Summary, it can be Seen that:

Firstly, the bufalin derivatives provided by the present invention, through structural modification of existing bufalin by introducing different phosphate ester groups, yield a new series of bufalin derivatives and their pharmaceutically acceptable salts. These derivatives not only exhibit excellent antitumor activity and low hERG potassium channel inhibitory activity but also significantly reduce toxic and side effects, making them suitable for the preparation of various antitumor drugs, cardiovascular and cerebrovascular disease treatments, and neurological disease treatments.

Secondly, the bufalin derivatives provided by the present invention offer a variety of selectable structural forms and their pharmaceutically acceptable salts. Therefore, they can meet the needs of preparing various pharmaceutical compositions and drug formulations, providing a foundation and ideas for developing new drug formulations with better therapeutic effects and lower toxic and side effects, and possessing broad application prospects.

In addition, the bufalin derivatives provided by the present invention have a simple and convenient preparation method, with inexpensive and easily obtainable raw materials, diverse selection options, and ease of industrial production. They can make significant contributions to safeguarding people's physical and mental health, and therefore have great value for promotion and application.

### In the Description Provided Above:

The terms such as “this example,” “examples of the present invention,” “as shown in . . . ”, “further,” and others are used to indicate that the specific features, structures, materials, or characteristics described in that example or example are included in at least one example or example of the present invention. In this specification, the illustrative expressions of these terms do not necessarily refer to the same example or example, and the specific features, structures, materials, or characteristics described can be combined or integrated in any one or more examples or examples in a suitable manner. Furthermore, without contradiction, ordinary technicians in the field can combine or integrate different examples or examples described in this specification, as well as the features of different examples or examples.

### Example 5

This example provides a bufalin phosphate derivative with the structure of formula IV.

### Case 13

This case provides a bufalin phosphate derivative with the structure shown in formula IVa.

The synthesis route provided is as follows:

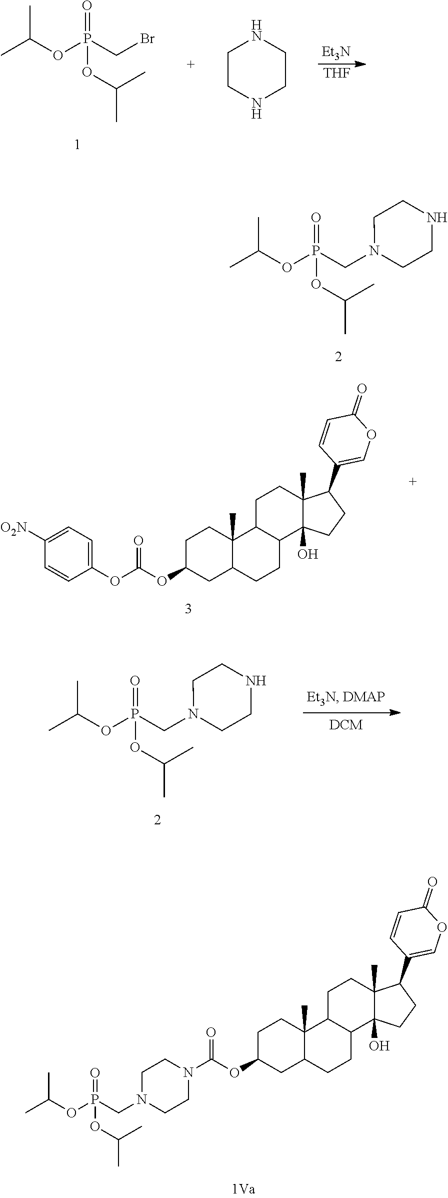

The specific process provided is: Bromomethyldiisopropyl phosphate 1 (130 mg, 0.5 mmol) and piperazine (301 mg, 3.5 mmol) are added to a flask. Under nitrogen protection, 10 mL of dried tetrahydrofuran is added and stirred to dissolve. Then, 0.1 mL of triethylamine is added, and the temperature is raised to reflux. After reacting for 8 hours, it is cooled to room temperature. After removing the solvent under reduced pressure, 5 mL of dichloromethane is added for dissolution. The organic phase is washed once with water and saturated saline solution, respectively. After drying with anhydrous sodium sulfate, the solvent is removed to obtain compound 2, which is directly used for the next step of the reaction.

After dissolving and stirring the previous product 2 (56 mg, 0.21 mmol) in 3 mL of dried dichloromethane, triethylamine (29 L, 0.21 mmol), DMAP (26 mg, 0.21 mmol), and p-nitrophenyl-3-bufalinyl carbonate 3 (40 mg, 0.07 mmol, prepared according to the literature by Min Lei, et al. Steroids, 2016, 108, 56-60) are added sequentially. The reaction is allowed to proceed at room temperature for 2 hours. The reaction mixture is washed once with a 10% aqueous citric acid solution, water, and saturated saline solution, respectively. After drying with anhydrous sodium sulfate, the solvent is removed under reduced pressure, and the product is subjected to column chromatography to obtain the white solid compound IVa with a yield of 62.9%.

### Test Results:

¹H NMR (600 MHz, Chloroform-d) 67.84 (dd, J=9.8, 2.6 Hz, 1H), 7.23 (dd, J=2.6, 1.1 Hz, 1H), 6.26 (dd, J=9.7, 1.1 Hz, 1H), 5.02 (d, J=3.9 Hz, 1H), 4.75 (q, J=6.5 Hz, 2H), 3.49 (s, 4H), 2.69 (d, J=65.2 Hz, 5H), 2.47 (dd, J=9.7, 6.5 Hz, 1H), 2.20 (dt, J=12.7, 9.3 Hz, 1H), 2.11-2.04 (m, 1H), 1.94-1.83 (m, 2H), 1.79-1.68 (m, 3H), 1.67-1.47 (m, 10H), 1.46-1.39 (m, 2H), 1.34 (dd, J=6.1, 1.5 Hz, 12H), 1.29 (d, J=4.9 Hz, 2H), 1.20 (dd, J=12.5, 3.6 Hz, 2H), 0.95 (s, 3H), 0.70 (s, 3H); HRMS (ESI, positive) m/z calcd for C₃₆H₅₇N₂O₈P [M+H]⁺: 677.3931; found 677.3945.

### Case 14

This case provides a bufalin phosphate derivative with the structure shown in formula IVb.

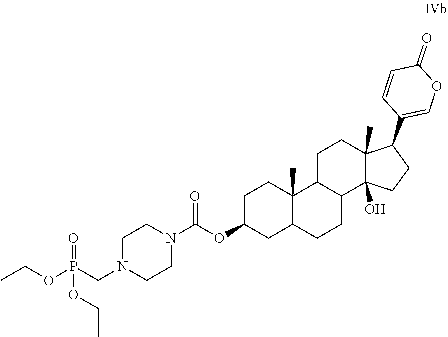 [SMILES: CCOP(=O)(CN1CCN(C(=O)O[C@H]2CC[C@@]3(C)C(CCC4C3CC[C@]3(C)[C@@H](c5ccc(=O)oc5)CC[C@]43O)C2)CC1)OCC]

The synthesis route and specific process for obtaining compound IVb are basically the same as those for obtaining compound IVa in Example 1. The only difference lies in the substitution of bromomethyldiethyl phosphate for compound 1 in Example 1. This substitution results in the obtainment of a white solid, namely compound IVb, with a molar yield of 68.2% relative to compound 3.

### Test Results:

¹H NMR (600 MHz, DMSO-d₆) δ7.93 (dd, J=9.8, 2.6 Hz, 1H), 7.52 (dd, J=2.6, 1.1 Hz, 1H), 6.29 (dd, J=9.7, 1.1 Hz, 1H), 4.90-4.82 (m, 1H), 4.15 (s, 1H), 4.09-3.97 (m, 4H), 3.35 (s, 3H), 2.80 (d, J=11.1 Hz, 2H), 2.52 (d, J=5.7 Hz, 4H), 2.46 (s, 1H), 2.04 (d, J=15.7 Hz, 2H), 1.96-1.87 (m, 1H), 1.77 (d, J=11.8 Hz, 2H), 1.66-1.44 (m, 8H), 1.43-1.29 (m, 4H), 1.23 (t, J=7.0 Hz, 9H), 1.10 (dtd, J=38.9, 12.8, 3.8 Hz, 2H), 0.89 (s, 3H), 0.59 (s, 3H); HRMS (ESI, positive) m/z calcd for C₃₄H₅₃N₂O₈P[M+H]⁺: 649.3618; found 649.3629.

### Case 16

This case provides a bufalin phosphate derivative with the structure shown in formula IVc.

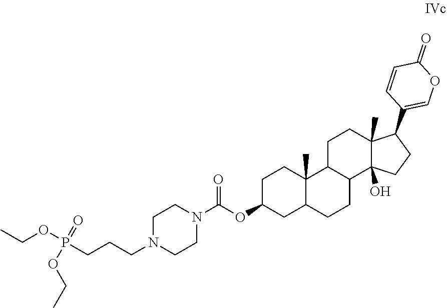 [SMILES: CCOP(=O)(CCCN1CCN(C(=O)O[C@H]2CC[C@@]3(C)C(CCC4C3CC[C@]3(C)[C@@H](c5ccc(=O)oc5)CC[C@]43O)C2)CC1)OCC]

The synthesis route and specific process for obtaining compound IVc are basically the same as those for obtaining compound IVa in Example 1. The only difference lies in the substitution of bromopropyldiethyl phosphate for compound 1 in Example 1. This substitution results in the obtainment of a white solid, namely compound IVc, with a molar yield of 58.7% relative to compound 3.

### Test Results:

¹H NMR (600 MHz, DMSO-d₆) δ7.93 (dd, J=9.7, 2.6 Hz, 1H), 7.52 (dd, J=2.6, 1.1 Hz, 1H), 6.29 (dd, J=9.7, 1.1 Hz, 1H), 4.89-4.82 (m, 1H), 4.15 (s, 1H), 4.02-3.91 (m, 4H), 3.35 (s, 4H), 2.45 (d, J=9.3 Hz, 1H), 2.39-2.25 (m, 6H), 2.11-1.99 (m, 2H), 1.92 (d, J=2.7 Hz, 1H), 1.82-1.68 (m, 4H), 1.65-1.46 (m, 10H), 1.43-1.29 (m, 4H), 1.22 (t, J=7.0 Hz, 7H), 1.18 (d, J=3.4 Hz, 1H), 1.17-1.05 (m, 2H), 0.89 (s, 3H), 0.59 (s, 3H); HRMS (ESI, positive) m/z calcd for C₃₆H₅₇N₂O₈P [M+H]⁺: 677.3931; found 677.3948.

It should be noted that, although this example only provides three specific bufalin phosphate derivatives with the structure of formula IV through Examples 1 to 3, namely those with the structures shown in formulas IVa, IVb, and IVc, those skilled in the art can fully understand that other specific bufalin phosphate derivatives with the structure of formula IV can also be obtained using methods similar to those described in Examples 1 to 3. For the sake of brevity in this specification, detailed descriptions of such derivatives are not provided.

### Example 6

This example provides pharmaceutically acceptable salts of bufalin phosphate derivatives with the structure shown in formula IV.

Firstly, in this example, the bufalin phosphate derivatives with structures shown in formulas IVa to IVc, obtained from the examples in Example 5, are respectively reacted with pharmaceutically acceptable inorganic acids or organic acids to obtain the corresponding pharmaceutically acceptable salts of the bufalin phosphate derivatives with structures shown in formulas IVa to IVc. Since the relevant reactions are basic and commonly used in this field, they are not described in detail here for brevity.

Secondly, following the method for obtaining the pharmaceutically acceptable salts of bufalin phosphate derivatives with structures shown in formulas IVa to IVc, other bufalin phosphate derivatives with the structure shown in formula IV and their pharmaceutically acceptable salts can also be obtained. Since this process is a basic and commonly used reaction in this field, further detailed descriptions are not provided here for brevity.

In the process of providing pharmaceutically acceptable salts of the aforementioned bufalin phosphate derivatives:

The inorganic acid can be any one of hydrochloric acid, hydrobromic acid, phosphoric acid, nitric acid, or sulfuric acid.

The organic acid can be any one of formic acid, acetic acid, propionic acid, succinic acid, 1,5-naphthalenedisulfonic acid, ascorbic acid, glycyrrhizic acid, glycyrrhetic acid, oleanolic acid, crataegic acid, ursolic acid, corosolic acid, betulinic acid, boswellic acid, oxalic acid, tartaric acid, lactic acid, salicylic acid, benzoic acid, valeric acid, diethylacetic acid, malonic acid, succinic acid (note: succinic acid is repeated here, presumably a typo or oversight; it should be omitted or replaced with another acid), fumaric acid, pimelic acid, adipic acid, maleic acid, malic acid, aminosulfonic acid, phenylpropionic acid, gluconic acid, ascorbic acid (note: ascorbic acid is repeated here, it should be omitted or considered in the context of another relevant acid if intended differently), nicotinic acid, isonicotinic acid, methanesulfonic acid, ethanesulfonic acid, p-toluenesulfonic acid, citric acid, or an amino acid.

It should be noted that, although this example only demonstrates the reaction of three specific bufalin phosphate derivatives with the structure of formula IV, namely those with structures shown in formulas IVa, IVb, and IVc, with pharmaceutically acceptable inorganic or organic acids to obtain the corresponding salts of bufalin phosphate derivatives with structures shown in formulas IVa, IVb, and IVc, those skilled in the art can fully understand that similar methods can be applied to obtain the corresponding pharmaceutically acceptable salts of various compounds with the structure of formula IV for other specific bufalin phosphate derivatives. However, for the sake of brevity in this specification, detailed descriptions of such reactions are not provided.

### Example 7

This example provides a pharmaceutical composition containing a bufalin phosphate derivative.

A therapeutically effective amount of the bufalin phosphate derivative with the structure shown in formula IV obtained from Example 5 above, or the pharmaceutically acceptable salt of the bufalin phosphate derivative with the structure shown in formula IV obtained from Example 6 above, or more specifically, a therapeutically effective amount of the bufalin phosphate derivatives with structures shown in formulas IVa to IVc provided in the examples of Example 5, or the pharmaceutically acceptable salts of the bufalin phosphate derivatives with structures shown in formulas IVa to IVc provided in Example 6, is mixed or dissolved with one or more pharmaceutically acceptable carriers, excipients, and adjuvants to obtain the corresponding pharmaceutical composition of each bufalin phosphate derivative or its pharmaceutically acceptable salt.

Since these operations are commonly used basic operations in this field, for the sake of brevity, this example does not provide detailed descriptions.

### Example 8

This example provides a bufalin phosphate derivative formulation.

Taking the pharmaceutical compositions of each bufalin phosphate derivative or its pharmaceutically acceptable salt provided in Example 7, and performing corresponding operations according to the preparation methods of existing pharmaceutical formulations, at least one type of pharmaceutical formulation can be obtained, including but not limited to injectables, powder injections, emulsions for injection, tablets, pills, capsules, pastes, creams, patches, liniments, powders, sprays, implants, drops, suppositories, ointments, or nano-formulations. Among them, the injectables can be at least one type of small volume injectables, medium volume injectables, or large volume injectables. The nano-formulations can be liposomes.

Since these operations are commonly used basic operations in this field, for the sake of brevity, this example does not provide detailed descriptions.

### Example 9

This example provides an application of bufalin phosphate derivatives.

The application described in this example is the use of a therapeutically effective amount of the bufalin phosphate derivative with the structure shown in formula IV provided in Example 5 or its pharmaceutically acceptable salt, for preparing at least one type of antitumor drug, cardiovascular and cerebrovascular disease treatment drug, or nervous system disease treatment drug. Specifically:

The antitumor drug is intended for the treatment of at least one type of malignant tumor growing in the esophagus, stomach, intestines, rectum, mouth, pharynx, larynx, lungs, colon, breast, uterus, endometrium, ovaries, prostate, testes, bladder, kidneys, liver, pancreas, bones, connective tissues, skin, eyes, brain, and central nervous system of humans or animals, as well as for the treatment of at least one disease including thyroid cancer, leukemia, Hodgkin's disease, lymphoma, and myeloma.

### Efficacy Example

This efficacy example is used to characterize the in vitro antitumor activity of the bufalin phosphate derivatives with structures shown in formulas IVa to IVc provided in Example 5, in order to realize the application described in Example 9.

Test Method: The CCK-8 method from “Methods for New Drug Pharmacological Research” edited by Lv Qiujun (2007: 242-243) was adopted.

Cell lines used: Human lung adenocarcinoma cells A549, human high-metastasis lung cancer cells 95D, and human colon cancer cells HCT116, which were cryopreserved and passaged by the Pharmacological Laboratory of the Shanghai Institute of Pharmaceutical Industry, were selected. The culture medium used was DMEM+10% FBS+antibiotics.

### In Vitro Activity Testing Process:

Cells in the logarithmic growth phase were taken and seeded into 96-well plates at a density of 7×10³ cells per well. They were then incubated at 37° C. in a 5% CO2 environment for 24 hours to maintain a normal physiological pH.

After cell adhesion, different concentrations of the test compounds, namely the bufalin derivatives IVa to IVc prepared in Example 5, as well as the comparative compound doxorubicin, were added to three replicate wells, with 0.1% DMSO added to separate wells as a control.

After 72 hours, 10 μL of the CCK-8 cell counting kit was added to each well, and the plates were incubated again at 37° C. for 0.5 to 1 hour. The absorbance (OD) was read at 450 nm on a full-function microplate reader, Biotek Synergy H2, and the concentration causing 50% inhibition of cell growth (IC₅₀) was calculated through simulation using GraphPad Prism software.

The detailed test results are shown in Table 5.

TABLE 5

| | IC₅₀ (μM) | | |
|---|---|---|---|
| compound | A549 | HCT116 | 95D |
| IVa | 0.019 | 0.117 | 0.574 |
| IVb | 0.018 | 0.040 | 0.262 |
| IVc | 0.011 | 0.027 | 0.072 |
| Doxorubicin | 0.008 | 0.019 | 0.115 |

The experimental results indicate that the bufalin phosphate derivatives with structures shown in formulas IVa to IVc provided in Example 5 exhibit excellent antitumor activity and show remarkable activity against lung cancer and colon cancer. Their activity against A549 is comparable to that of the positive control drug doxorubicin. The bufalin phosphate derivatives with structures shown in formulas IVb and IVc also demonstrate comparable activity against HCT116 to doxorubicin, while the bufalin phosphate derivative with the structure shown in formula IVc exhibits superior activity against 95D compared to doxorubicin.

It should be noted that although Table 5 only lists the in vitro antitumor activity test data for the bufalin phosphate derivatives with structures shown in formulas IVa to IVc provided in Example 5, based on similar experiments, it can be concluded that the bufalin phosphate derivatives with the structure shown in formula IV provided in Example 5 or their pharmaceutically acceptable salts provided in Example 2 all possess antitumor activity similar to that of the bufalin phosphate derivatives with structures shown in formulas IVa to IVc. They also show remarkable activity against lung cancer and colon cancer, with their activity against A549 being comparable to that of the positive control drug doxorubicin. The bufalin phosphate derivatives with structures shown in formulas IVb and IVc also demonstrate comparable activity against HCT116 to doxorubicin, while the bufalin phosphate derivative with the structure shown in formula IVc exhibits superior activity against 95D compared to doxorubicin. Therefore, it can be considered that the bufalin phosphate derivatives and their salts provided by the present invention can be used for preparing antitumor drugs, realizing the application described in Example 9.

In summary, it can be seen that: Firstly, the bufalin phosphate derivatives provided by the present invention are a new series of bufalin phosphate derivatives and their pharmaceutically acceptable salts obtained by introducing different phosphate ester groups into the existing bufalin structure. These derivatives exhibit excellent antitumor activity and can be used for preparing various antitumor drugs, cardiovascular and cerebrovascular disease treatment drugs, as well as nervous system disease treatment drugs.

Secondly, the bufalin phosphate derivatives provided by the present invention have a variety of selectable structural forms and their pharmaceutically acceptable salts. Therefore, they can meet the needs of preparing various pharmaceutical compositions, drug formulations, etc. They provide a foundation and ideas for preparing new drug formulations with better therapeutic effects and lower toxic and side effects, and have broad application prospects.

Lastly, it should be noted that: The above examples are merely used to illustrate the technical solutions of the present invention, rather than to limit them. Although the present invention has been described in detail with reference to the preceding examples or examples, ordinary technicians in the field should understand that they can still modify the technical solutions recorded in the preceding examples or examples, or make equivalent substitutions to some or all of the technical features therein. These modifications or substitutions do not deviate the essence of the corresponding technical solutions from the scope of the technical solutions of the examples or examples of the present invention. Non-essential improvements, adjustments, or substitutions made by technicians in the field based on the content of this specification fall within the scope of protection claimed by the present invention.

What is claimed is:

1. A bufalin derivative, wherein the bufalin derivative is a compound having a structure represented by Formula I, Formula II, or Formula III, or a pharmaceutically acceptable salt thereof: 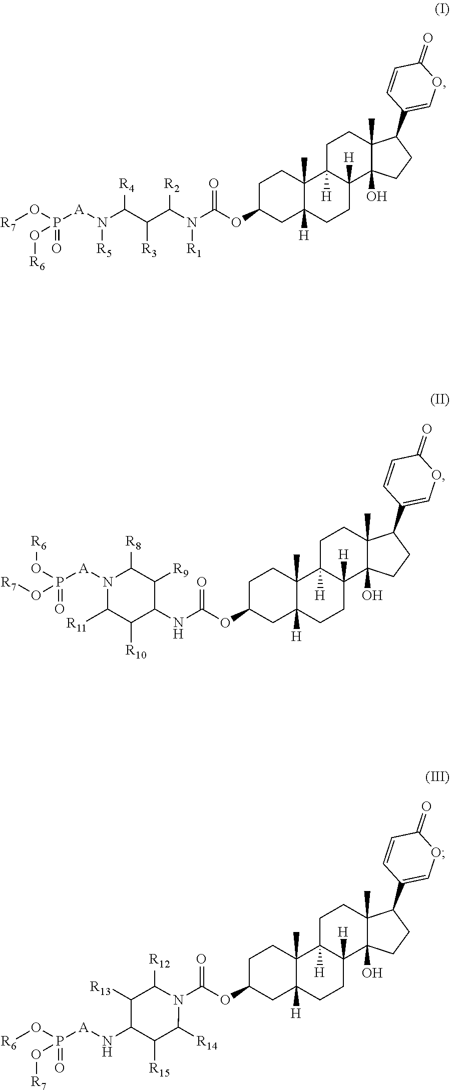
wherein:
R₁ is selected from the group consisting of hydrogen, deuterium, and substituted and unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl;
R₂ and R₃ are each independently selected from the group consisting of hydrogen, deuterium, hydroxyl, halogen, and substituted and unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl.
R₄ and R₁ together form a substituted or unsubstituted nitrogen-containing heterocyclic ring;
R₅ is selected from the group consisting of hydrogen, deuterium, and substituted and unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl, or R₅ and R₁ together form a substituted or unsubstituted nitrogen-containing heterocyclic ring;
R₆ and R₇ are each independently selected from the group consisting of hydrogen, and substituted and unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl;
R₈, R₉, R₁₀, and Ru are each independently selected from the group consisting of hydrogen, deuterium, hydroxyl, halogen, and substituted and unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl;
R₁₂, R₁₃, R₁₄, and R₁₅ are each independently selected from the group consisting of hydrogen, deuterium, hydroxyl, halogen, and substituted and unsubstituted alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl; and
A is a linear or branched C₁-C₆ alkane.

2. The bufalin derivative according to claim 1, wherein in the compound represented by Formula II or Formula III, or a pharmaceutically acceptable salt thereof:
R₆ and R₇ are each independently selected from the group consisting of a 3- to 8-membered cycloalkyl, a C₆-C₁₂ aryl, and any one of a C1- to C8-alkyl, a 3- to 8-membered cycloalkyl-C1- to C8-alkyl, a 3- to 8-membered heterocyclyl-C1- to C8-alkyl, a 3- to 8-membered alkyl containing an ether bond, or a 3- to 8-membered alkyl containing a thioether bond, each of which is optionally substituted with one or more T groups;
R₈, R₉, R₁₀, and Ru are each independently selected from a 3- to 8-membered cycloalkyl, a 3- to 8-membered alkyl containing a thioether bond, and any one of a C1- to C8-alkyl, a 3- to 8-membered cycloalkyl-C1- to C8-alkyl, a 3- to 8-membered heterocyclyl-C1- to C8-alkyl, a 3- to 8-membered alkyl containing an ether bond, or a 3- to 8-membered alkyl containing a thioether bond, each of which is optionally substituted with one or more T groups;
R₁₂, R₁₃, R₁₄, and R₁₅ are each independently selected from a 3- to 8-membered cycloalkyl, a 3- to 8-membered alkyl containing a thioether bond, and any one of a C1- to C8-alkyl, a 3- to 8-membered cycloalkyl-C1- to C8-alkyl, a 3- to 8-membered heterocyclyl-C1- to C8-alkyl, a 3- to 8-membered alkyl containing an ether bond, or a 3- to 8-membered alkyl containing a thioether bond, each of which is optionally substituted with one or more T groups;
T group is selected from the group consisting of F, Cl, Br, I, OH, OCH₃, OCH₂CH₃, SCH₃, SCH₂CH₃, NHBoc, NHC(═O)CH₃, NHC(═O)CH₂CH₃, C(═O)NH₂, C(═O)OC(CH₃)₃, C(═O)OCH₂CH₃, CH₂F, CHF₂, and CF₃.

3. The bufalin derivative according to claim 1, wherein in the compound represented by Formula II or Formula III, or a pharmaceutically acceptable salt thereof:
R₆ and R₇ are each independently selected from the group consisting of a 3- to 8-membered cycloalkyl and a C6- to C12-aryl, each of which is optionally substituted with one or more T groups;
R₈, R₉, R₁₀, and R₁₁ are each independently selected from a 3- to 8-membered alkyl containing an ether bond, each of which is optionally substituted with one or more T groups;
R₁₂, R₁₃, R₁₄, and R₁₅ are each independently selected from a 3- to 8-membered alkyl, each of which is optionally substituted with one or more T groups;
T group is selected from the group consisting of F, Cl, Br, I, OH, OCH₃, OCH₂CH₃, SCH₃, SCH₂CH₃, NHBoc, NHC(═O)CH₃, NHC(═O)CH₂CH₃, C(═O)NH₂, C(═O)OC(CH₃)₃, C(═O)OCH₂CH₃, CH₂F, CHF₂, and CF₃.

4. The bufalin derivative according to claim 1, wherein the bufalin derivative is selected from the group consisting of Formulas 1a to 1l, and pharmaceutically acceptable salts thereof:

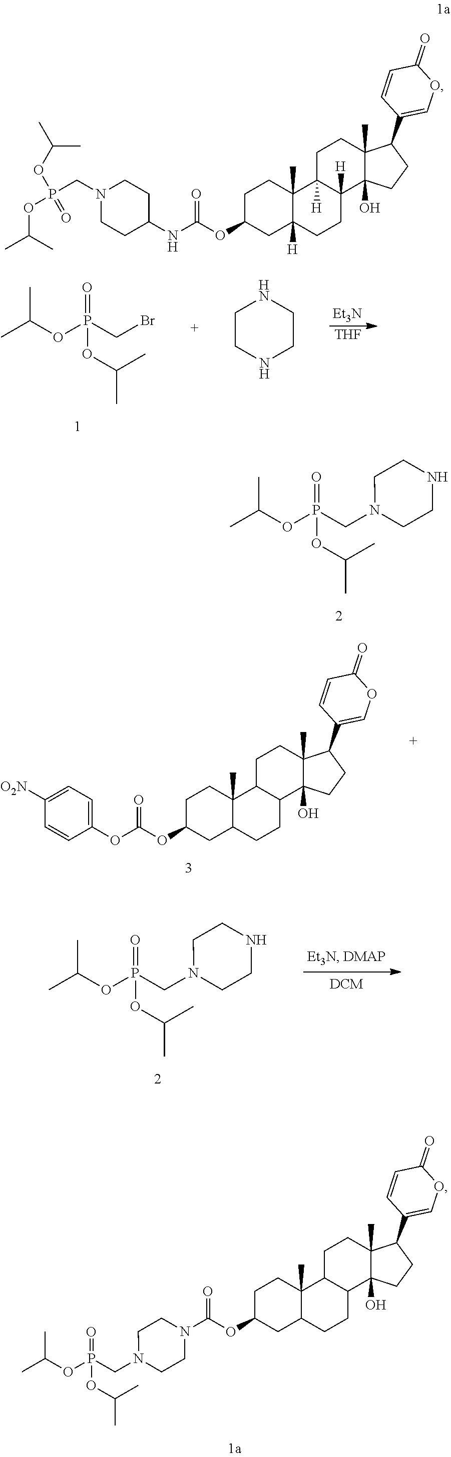

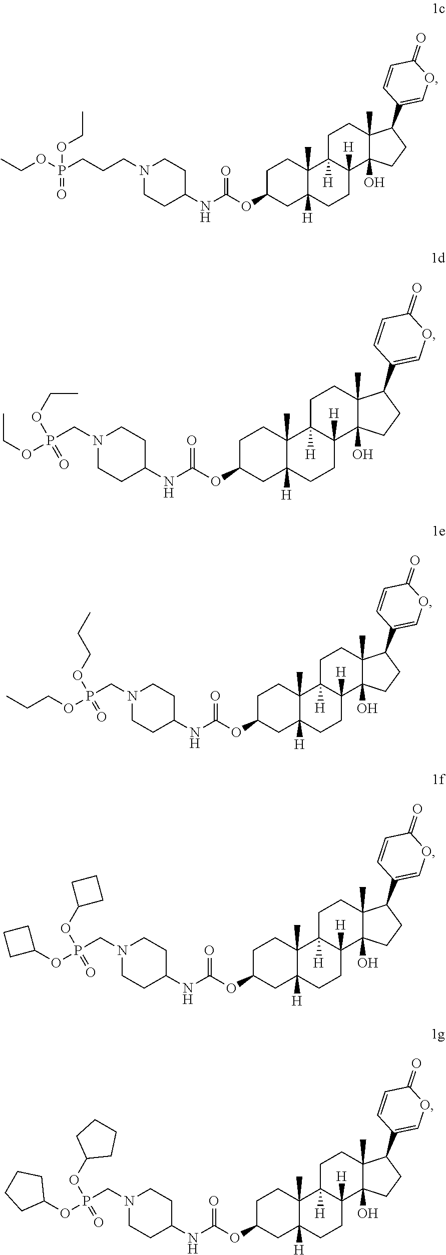

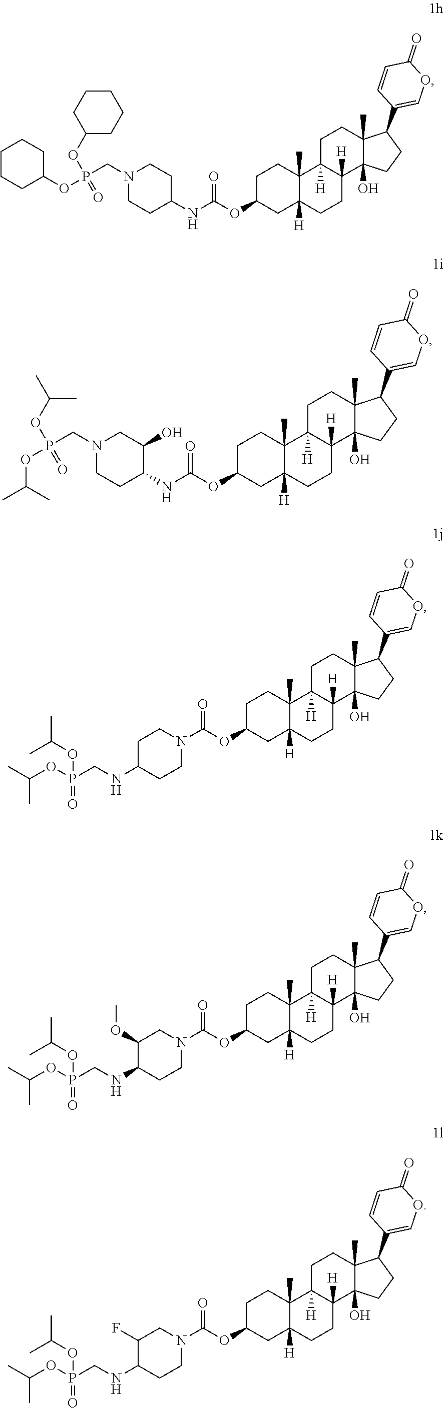

5. The bufalin derivative according to claim 1, wherein the pharmaceutically acceptable salt is a salt formed from reaction of the compound with a pharmaceutically acceptable inorganic acid or organic acid.

6. The bufalin derivative according to claim 5, wherein the pharmaceutically acceptable inorganic acid is selected from the group consisting of hydrochloric acid, hydrobromic acid, phosphoric acid, nitric acid, and sulfuric acid;
the pharmaceutically acceptable organic acid is selected from the group consisting of formic acid, acetic acid, propionic acid, succinic acid, 1,5-naphthalenedisulfonic acid, asiatic acid, glycyrrhizic acid, glycyrrhetic acid, oleanolic acid, crataegic acid, ursolic acid, corosolic acid, betulinic acid, boswellic acid, oxalic acid, tartaric acid, lactic acid, salicylic acid, benzoic acid, valeric acid, diethylacetic acid, malonic acid, succinic acid, fumaric acid, pimelic acid, adipic acid, maleic acid, malic acid, aminosulfonic acid, phenylpropionic acid, gluconic acid, ascorbic acid, nicotinic acid, isonicotinic acid, methanesulfonic acid, ethanesulfonic acid, p-toluenesulfonic acid, citric acid, and an amino acid.

7. A method for preparing a bufalin derivative represented by Formula I, wherein the method comprises step e, steps a to b and e, or steps c to d and e in the following synthetic route: 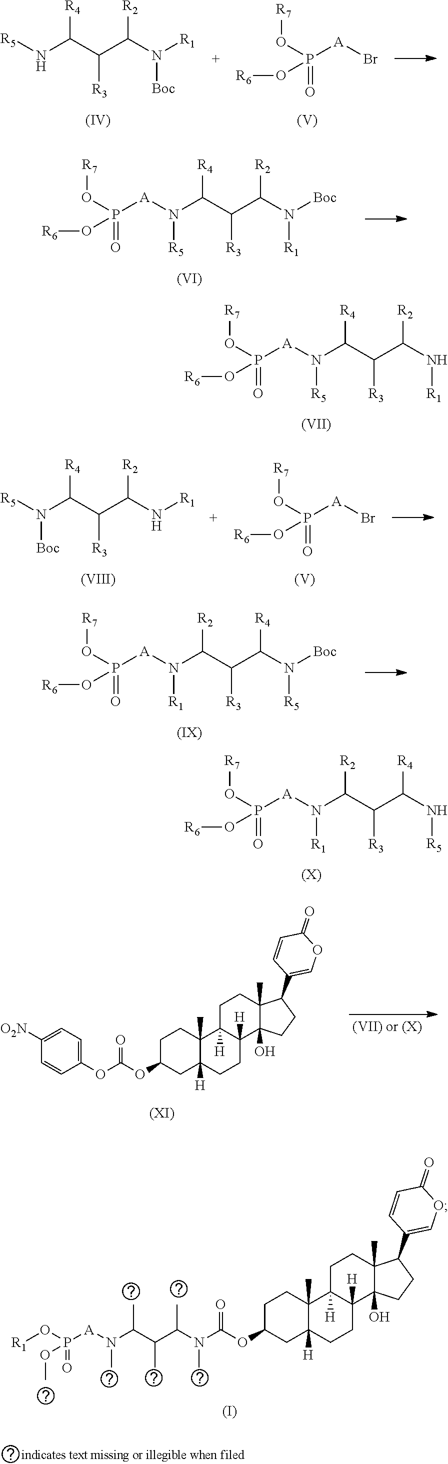
step (a) reacting compound IV with compound V in an organic solvent containing a first alkaline catalyst to produce compound VI, wherein a molar ratio of the compound IV to the compound V to the first alkaline catalyst is 1: (1-3): (1-3);
step (b) subjecting the compound VI to catalytic reaction in the presence of an acidic catalyst in an organic solvent to produce compound VII;
step (c) reacting compound VIII with compound V in an organic solvent containing a first alkaline catalyst to produce compound IX, wherein a molar ratio of the compound VIII to the compound V to the first alkaline catalyst is 1: (1-3): (1-3);
step (d) subjecting the compound IX to catalytic reaction in the presence of an acidic catalyst in an organic solvent to produce compound X; and
step (e) reacting the compound XI with the compound VII or the compound X in an organic solvent containing a second alkaline catalyst to produce compound I, wherein a molar ratio of the compound XI to the compound VII or the compound X to the second alkaline catalyst is 1: (1-5): (1-5).

8. The method according to claim 7, wherein the first alkaline catalyst is an inorganic alkaline compound, the second alkaline catalyst is a nitrogen-containing compound, and the acidic catalyst is an inorganic acid or an organic acid.

9. The method according to claim 8, wherein the inorganic alkaline compound is selected from the group consisting of sodium carbonate, potassium carbonate, cesium carbonate and a combination thereof;
the nitrogen-containing compound is selected from the group consisting of 4-dimethylaminopyridine, triethylamine, pyridine and a combination thereof,
the acidic catalyst is selected from the group consisting of hydrochloric acid, sulfuric acid, trifluoroacetic acid, acetic acid, or formic acid; and
the organic solvent is selected from the group consisting of dichloromethane, dichloroethane, ethyl acetate, acetonitrile, tetrahydrofuran and a combination thereof.

10. A pharmaceutical composition, comprising:
a therapeutically effective amount of the bufalin derivative of claim 1, or a pharmaceutically acceptable salt thereof, and
one or more of pharmaceutically acceptable carriers, excipients, and adjuvants.

11. A pharmaceutical formulation, wherein the pharmaceutical formulation is at least one of an injection, a powdered injection, an emulsion for injection, a tablet, a pill, a capsule, an ointment, a cream, a patch, a liniment, a powder, a spray, an implant, a drop, a suppository, a paste, and a nano-formulation prepared from the pharmaceutical composition of claim 10.

12. The pharmaceutical formulation according to claim 11, wherein the injection is at least one of a small-volume injection, a medium-volume injection, and a large-volume injection, and the nano-formulation is a liposome.

13. A method for treating at least one of a tumor disease, a cardiovascular and cerebrovascular disease and a neurological disease in a subject in need thereof, comprising:
administering a therapeutically effective amount of the bufalin derivative of claim 1 or a pharmaceutically acceptable salt thereof to the subject.

14. The method according to claim 13, wherein the tumor disease is selected from the group consisting of malignant tumors growing in the esophagus, stomach, intestine, rectum, mouth, pharynx, larynx, lungs, colon, breasts, uterus, endometrium, ovaries, prostate, testes, bladder, kidneys, liver, pancreas, bones, connective tissues, skin, eyes, brain, central nervous system of humans or animals, thyroid cancer, leukemia, Hodgkin's disease, lymphoma, myeloma and a combination thereof.

15. The method according to claim 13, wherein the tumor disease is tumor metastasis.

16. A bufalin phosphate derivative, wherein the bufalin phosphate derivative is a compound represented by formula IV or a pharmaceutically acceptable salt thereof: 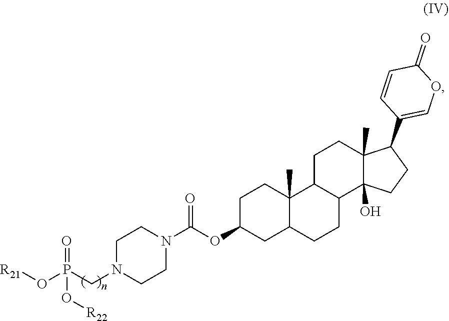
wherein:
R₂₁ and R₂₂ are each independently selected from the group consisting of hydrogen, deuterium, alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl; n is an integer from 1 to 10.

17. The bufalin phosphate derivative according to claim 16, wherein R₂₁ and R₂₂ are each independently selected from the group consisting of hydrogen, deuterium, alkyl, cycloalkyl, and aryl, and n is an integer from 1 to 3.

18. The bufalin phosphate derivative according to claim 17, wherein the bufalin phosphate derivative is a compound represented by formula IVa, IVb, or IVc, or a pharmaceutically acceptable salt thereof.

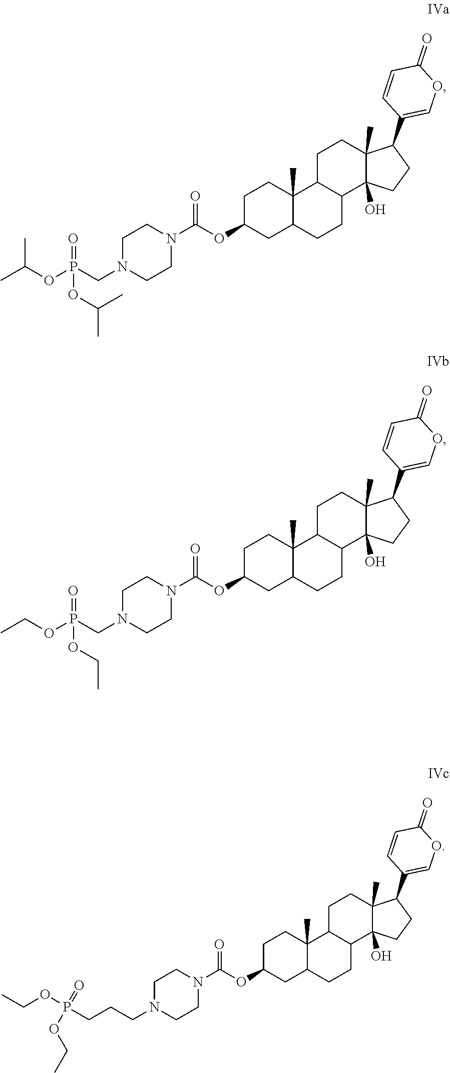 [SMILES: CC(C)OP(=O)(CN1CCN(C(=O)O[C@H]2CC[C@@]3(C)C(CCC4C3CC[C@]3(C)[C@@H](c5ccc(=O)oc5)CC[C@]43O)C2)CC1)OC(C)C ; CCOP(=O)(CN1CCN(C(=O)O[C@H]2CC[C@@]3(C)C(CCC4C3CC[C@]3(C)[C@@H](c5ccc(=O)oc5)CC[C@]43O)C2)CC1)OCC ; CCOP(=O)(CCCN1CCN(C(=O)O[C@H]2CC[C@@]3(C)C(CCC4C3CC[C@]3(C)[C@@H](c5ccc(=O)oc5)CC[C@]43O)C2)CC1)OCC]

19. The bufalin phosphate derivative according to claim 16, wherein the pharmaceutically acceptable salt is a salt formed from reaction of the compound with a pharmaceutically acceptable inorganic acid or organic acid;
the pharmaceutically acceptable inorganic acid is hydrochloric acid, hydrobromic acid, phosphoric acid, nitric acid, or sulfuric acid; and
the pharmaceutically acceptable organic acid is selected from the group consisting of formic acid, acetic acid, propionic acid, succinic acid, 1,5-naphthalenedisulfonic acid, ascorbic acid, glycyrrhizic acid, glycyrrhetic acid, oleanolic acid, crataegic acid, ursolic acid, corosolic acid, betulinic acid, boswellic acid, oxalic acid, tartaric acid, lactic acid, salicylic acid, benzoic acid, pentanoic acid, diethylacetic acid, malonic acid, succinic acid, fumaric acid, pimelic acid, adipic acid, maleic acid, malic acid, aminosulfonic acid, phenylpropionic acid, gluconic acid, ascorbic acid, nicotinic acid, isonicotinic acid, methanesulfonic acid, ethanesulfonic acid, p-toluenesulfonic acid, citric acid, and an amino acid.

20. The bufalin phosphate derivative according to claim 16, wherein the alkyl group is a monovalent saturated linear or branched alkyl group consisting of one to twelve carbon atoms, comprising methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, cyclopropyl, cyclobutyl, cyclopentyl, cyclohexyl, cycloheptyl, and cyclooctyl; and the hydrogen atoms in the alkyl group can also optionally be independently substituted by one or more substituents;
the alkenyl group is a monovalent linear or branched alkenyl group consisting of two to twelve carbon atoms with at least one carbon-carbon sp² double bond, comprising vinyl, allyl, 1-cyclopent-1-enyl, 1-cyclopent-2-enyl, 1-cyclopent-3-enyl, 5-hexenyl, 1-cyclohex-2-enyl, 1-cyclohex-2-enyl (note: there may be a typo in the original text as 1-cyclohex-2-enyl is repeated; possibly intended as another isomer like 1-cyclohex-3-enyl or similar), and 1-cyclohex-3-enyl; and the hydrogen atoms in the alkenyl group can also optionally be independently substituted by one or more substituents, including “cis” and “trans” orientations or “E” and “Z” orientations;
the alkynyl group is a monovalent linear or branched alkynyl group consisting of two to twelve carbon atoms with at least one carbon-carbon sp³ triple bond, comprising ethynyl and propynyl; and
hydrogen atoms in the alkynyl group can also optionally be independently substituted by one or more substituents;
the heteroalkyl group is a monovalent saturated linear or branched alkyl group consisting of one to twelve carbon atoms, wherein at least one carbon atom is replaced by a nitrogen, oxygen, or sulfur atom, and these nitrogen, oxygen, or sulfur atoms can appear in the middle or at the terminus of the heteroalkyl group, which can be a carbon-based group or a heteroatom-based group; and
hydrogen atoms in the heteroalkyl group can also optionally be independently substituted by one or more substituents; and the heteroalkyl group also includes alkoxy and heteroalkoxy groups;
the heteroalkenyl group is a monovalent linear or branched alkenyl group consisting of two to twelve carbon atoms and containing at least one double bond, wherein at least one carbon atom is replaced by a nitrogen, oxygen, or sulfur atom, and these nitrogen, oxygen, or sulfur atoms can appear in the middle or at the terminus of the heteroalkenyl group, which can be a carbon-based group or a heteroatom-based group; and the hydrogen atoms in the heteroalkenyl group can also optionally be independently substituted by one or more substituents, including those with “cis” and “trans” orientations or “E” and “Z” orientations;
the heteroalkynyl group is a monovalent linear or branched alkynyl group consisting of two to twelve carbon atoms and containing at least one triple bond, wherein at least one carbon atom is replaced by a nitrogen, oxygen, or sulfur atom, and these nitrogen, oxygen, or sulfur atoms can appear in the middle or at the terminus of the heteroalkynyl group, which can be a carbon-based group or a heteroatom-based group; and the hydrogen atoms in the heteroalkynyl group can also optionally be independently substituted by one or more substituents;
the cycloalkyl group is a monovalent non-aromatic saturated or partially saturated cyclic hydrocarbon radical having three to ten carbon atoms, as well as cyclic hydrocarbon radicals with bicyclic, tricyclic, or polycyclic structures, including cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclodecyl, and cyclododecyl; and cyclic hydrocarbon radicals with bicyclic, tricyclic, or polycyclic structures can also optionally be fused with saturated or partially unsaturated cycloalkyl or heterocyclic radicals or aryl or heteroaryl rings to form saturated or partially unsaturated cycloalkyl groups, wherein bicyclic carbon rings with 7 to 12 atoms can be bicyclo[4,5], [5,5], [5,6], or [6,6] systems, or bridged systems including bicyclo[2.2.1]heptane, bicyclo[2.2.2]octane, and bicyclo[3.2.2]nonane;
the heterocyclic radical is a saturated or partially unsaturated carbocyclic radical having 3 to 8 ring atoms, wherein at least one ring atom is a nitrogen, oxygen, or sulfur atom, and the remaining ring atoms are carbon atoms, and wherein one or more of these ring atoms can also optionally be independently substituted by one or more substituent groups, which can be carbon-based or heteroatom-based groups; and the heterocyclic radical also includes heterocyclic alkoxy groups and heterocyclic radicals fused with saturated, partially unsaturated, or fully unsaturated (i.e., aromatic) carbocyclic or heterocyclic rings, with the heterocyclic ring system including pyrrolidinyl, tetrahydrofuranyl, dihydrofuranyl, tetrahydrothiophenyl, tetrahydropyranyl, dihydropyranyl, tetrahydrothiopyranyl, piperidinyl, morpholinyl, 4-thiomorpholinyl, thioxanyl, piperazinyl, homopiperazinyl, azetidinyl, oxetanyl, thietanyl, homopiperidinyl, oxoheptanyl, thioheptanyl, oxazepinyl, diazepinyl, thiazepinyl, 2-pyrrolinyl, 3-pyrrolinyl, dihydroindolyl, 2H-pyranyl, 4H-pyranyl, dioxanyl, 1,3-dioxolanyl, pyrazolinyl, dithianyl, dihydropyranyl, dihydrothiophenyl, dihydrofuranyl, pyrazolidinyl, imidazolinyl, imidazolidinyl, 3-azabicyclo[3.1.0]hexane, 3-azabicyclo[4.1.0]heptane, azabicyclo[2.2.2]hexane, 3H-indolyl, quinoxalinyl, and N-pyridinylurea; and the heterocyclic radical also includes spirocyclic rings and N-linked pyrrol-1-yl, imidazol-1-yl, or C-linked pyrrol-3-yl, imidazol-3-yl, as well as dihydroisoindole-1,3-dionyl and 1,1-dioxothiomorpholinyl;
the cycloalkylalkyl group is an alkyl group substituted by a cycloalkyl moiety, including cyclopropylmethyl and five- or six-membered cycloalkyl-C1-3-alkyl groups;
the aryl group is an optionally substituted monocyclic or polycyclic group or ring system containing at least one aromatic hydrocarbon ring, including phenyl, naphthyl, fluorenyl, azulenyl, anthryl, phenanthryl, pyrenyl, biphenyl, and terphenyl;
the arylalkyl group is an alkyl group substituted by one or more aryl moieties, including aryl-C1-3-alkyl groups such as benzyl and phenylethyl;
the heteroaryl group is an optionally substituted monocyclic or polycyclic group or ring system containing at least one aromatic ring with one or more nitrogen, oxygen, or sulfur atoms, wherein:
monocyclic heteroaryl groups include furanyl, imidazolyl, isothiazolyl, isoxazolyl, oxadiazolyl, oxazolyl, pyrazinyl, pyrazolyl, pyridazinyl, pyridinyl, pyrimidinyl, pyrrolyl, thiadiazolyl, thiazolyl, thiophenyl, tetrazolyl, triazinyl, and triazolyl;
bicyclic heteroaryl groups include benzofuranyl, benzimidazolyl, benzoisoxazolyl, benzopyranyl, benzothiadiazolyl, benzothiazolyl, benzothiophenyl, benzotriazolyl, benzoxazolyl, furano[2,3-b]pyridinyl, imidazo[4,5-b]pyridinyl, imidazo[2,1-b]thiazolyl, indolinyl, indolyl, indazolyl, isobenzofuranyl, isobenzothiophenyl, isoindolyl, isoquinolinyl, isothiazolyl, naphthyridinyl, oxazolo[4,5-b]pyridinyl, phthalazinyl, pteridinyl, purinyl, pyrido[3,4-b]pyridinyl, pyrrolo[2,3-b]pyridinyl, quinolinyl, quinoxalinyl, quinazolinyl, thiadiazolo[4,5-b]pyrimidinyl, and thiopheno[2,3-b]pyridinyl;
tricyclic heteroaryl groups include acridinyl, benzindolyl, carbazolyl, dibenzofuranyl, phenanthridinyl, phenanthrolinyl, phenanthridinyl, phenoxazinyl, phenazinyl, phenothiazinyl, phenoxazinyl, and xanthinyl;
the heteroarylalkyl group is an alkyl group substituted by a heteroaryl moiety, specifically including five- or six-membered heteroaryl-C1-3-alkyl groups such as oxazolylmethyl and pyridinylethyl; and
the heterocyclic radical alkyl group is an alkyl group substituted by a heterocyclic radical moiety, specifically including five- or six-membered heterocyclic radical-C1-3-alkyl groups such as tetrahydropyranylmethyl.

21. The bufalin phosphate derivative according to claim 16, wherein one or more hydrogen atoms in each of the alkyl, alkenyl, alkynyl, heteroalkyl, heteroalkenyl, heteroalkynyl, cycloalkyl, heterocyclyl, cycloalkylalkyl, aryl, arylalkyl, heteroaryl, heteroarylalkyl, and heterocyclylalkyl groups are independently substituted by substituents selected from the group consisting of F, Cl, Br, I, CN, CF₃, OR, R, ═O, ═S, ═NR, ═N+(O)(R), ═N(OR), =N+(O)(OR), ═N—NRR′, —C(═O)R, —C(═O)OR, —C(═O)NRR′, —NRR′, —N+RR′R″, —N(R)C(═O)R′, —N(R)C(═O)OR′, —N(R)C(═O)NR′R″, —SR, —OC(═O)R, —OC(═O)OR, —OC(═O)NR′R″, —OS(O)₂OR, —OP(═O)(OR)₂, —OP(OR)₂, —P(═O)(OR)₂, —P(═O)(OR)NR′R″, —S(O)R, —S(O)₂R, —S(O)₂NR, —S(O)(OR), —S(O)₂(OR), —SC(═O)R, —SC(═O)OR, ═O, and —SC(═O)NR′R″, wherein R, R′, and R″ are each independently selected from hydrogen, deuterium, alkyl, alkenyl, alkynyl, aryl, and heterocyclyl.

22. A pharmaceutical composition, comprising:
a therapeutically effective amount of the bufalin phosphate derivative of claim 16; and
one or more of pharmaceutically acceptable carriers, excipients, and adjuvants.

23. A bufalin phosphate derivative formulation, wherein the bufalin phosphate derivative formulation is at least one of an injection, a powdered injection, an emulsion for injection, a tablet, a pill, a capsule, a paste, a cream, a patch, a liniment, a powder, a spray, an implant, a drop, a suppository, an ointment, and a nano-formulation prepared from the pharmaceutical composition according to claim 22, wherein the injection is at least one of a small volume injection, a medium volume injection, or a large volume injection, and the nano-formulation is a liposome.

24. A method for treating at least one of a tumor disease, a cardiovascular and cerebrovascular disease and a neurological disease in a subject in need thereof, comprising:
administering a therapeutically effective amount of the bufalin phosphate derivative according to claim 16 to the subject.

25. The method according to claim 24, wherein the tumor disease is selected from the group consisting of malignant tumors growing in esophagus, stomach, intestine, rectum, mouth, pharynx, larynx, lungs, colon, breasts, uterus, endometrium, ovaries, prostate, testes, bladder, kidneys, liver, pancreas, bones, connective tissues, skin, eyes, brain, central nervous system of humans or animals, thyroid cancer, leukemia, Hodgkin's disease, lymphomas, and myelomas.
